(19) United States
(12) Patent Application Publication
(10) Pub. No.: US 2025/0122672 A1
(43) Pub. Date: Apr. 17, 2025

(54) BIODEGRADABLE CONTAINER WITH A PERSONAL CARE COMPOSITION

(71) Applicant: The Procter & Gamble Company, Cincinnati, OH (US)

(72) Inventors: Stefano Bartolucci, BAD HOMBURG VOR DER HÖHE (DE); Emily Charlotte Boswell, CINCINNATI, OH (US); Patti Jean Kellett, CINCINNATI, OH (US); Jennifer Flohr, SCHWALBACH AM TAUNUS (DE); Jodi Lee Brown, CINCINNATI, OH (US); Valerie Wong, MASON, OH (US); Jessa Leigh Meyers, CINCINNATI, OH (US); Robert Michael Pettifer, Cincinnati, OH (US)

(21) Appl. No.: 18/913,075

(22) Filed: Oct. 11, 2024

Related U.S. Application Data
(60) Provisional application No. 63/589,446, filed on Oct. 11, 2023.

(51) Int. Cl. D21J 1/08; A45D 34/00; A61K 8/20; A61K 8/34; A61K 8/49; A61K 8/73; A61Q 5/00; A61Q 5/02; B32B 27/10; B32B 27/36; B65D 23/02; B65D 25/14; B65D 65/46; D21H 19/14; D21H 19/18; D21H 19/34; D21H 19/82

(52) U.S. Cl. CPC D21J 1/08; A45D 34/00; A61K 8/20; A61K 8/345; A61K 8/4926; A61K 8/4946; A61K 8/731; A61Q 5/006; A61Q 5/02; B32B 27/10; B32B 27/36; B65D 23/02; B65D 25/14; B65D 65/466; D21H 19/14; D21H 19/18; D21H 19/34; D21H 19/824; A61K 2800/87; B32B 2250/02; B32B 2307/7163; B32B 2307/738; B32B 2439/60; B32B 2439/62; B65D 2565/387

(57) ABSTRACT

The present invention is directed to a biodegradable container in combination with a liquid personal care composition comprising: a biodegradable vessel having at least one opening and a biodegradable cover for the opening wherein the container, a liquid personal care composition comprising from about 14% to about 50% water; from about 20% to about 70% of a humectant; wherein there is a Water Activity (Aw) of about 0.40 to about 0.90.

### FIELD OF THE INVENTION

The invention relates to a biodegradable container including a biodegradable vessel having at least one opening, a biodegradable cover for the opening, the vessel having a biodegradable coating with a liquid personal care composition having lower water activity and lower water content. The invention also relates to a method for producing the container.

### BACKGROUND OF THE INVENTION

Since the era of industrialization, plastic products have been widely used in daily life. By virtue of quite low production costs and their versatility, plastic packaging materials have a higher rate of increase in the world market compared with other packaging materials. However, most of the plastics that we have on the market are made of virgin crude oil which is not a renewable source. Despite continuous improvement in the waste management infrastructure, plastic packing sometimes doesn't get recycled after use and as a result, leak into the environment where it can be very persistent. Also, small plastic containers-such as those used for trial or sample sizes—can be difficult to recycle due to their small size and relatively low intrinsic recovery value. Thus, these containers can be diverted to undesired pathways such as incineration or landfill. Plastic pollution is causing both increased scrutiny by society in the use of plastics as well as an emergence of new environmental regulations limiting the use of plastics in packaging especially for applications with a short-life span.

Packaging made from natural cellulose fibers has become a point of increasing interest as part of a general movement towards inclusion of renewal and less persistent feedstock. Fiber-based packaging generally also has a very high rate of recyclability. Cellulosic articles are generally formed as film or multi-ply boards using paper making processes or as 3D formed objects using pulp molding methods. While fibers can provide excellent structural support and a good decoration surface, paper sheets or formed objects alone cannot be used to pack liquid products due to their poor oxygen and moisture barrier and poor liquid containment properties leading to integrity failures. Thus, a protective coating is generally applied on the inner side after the cellulosic article is manufactured to extend the shelf life of the packaged liquid products.

Liquid packaging boards (LPB) are generally laminated with polymers with heat seal properties such as PE in a structure that can include one or more barrier layers such as EVOH, vacuum metalized aluminum oxide, etc. or it may be coated with thin layer applied using a dispersion technique such as spray, roll, dip, blade or curtain coating. However, such coatings bring trade-offs between barrier performance, recyclability, and package biodegradability. In the present invention, a pulp vessel may be manufactured using rigid pulp molding. To achieve the desired liquid containment, moisture and oxygen barrier properties, such vessels can be coated by either a thin plastic liner or by spray coating. Like paper sheets, the inclusion of such liners, coating and additives come with compromises between either barrier performance, recyclability, and package biodegradability. Further, high fiber inclusion in functional appendices such as necks and closures present challenges due to high forming tolerances requirements and coating integrity reliability from multi-open/close cycles.

In the present invention, packages made from liquid carton boards and rigid pulp molding can include one or more biodegradable and/or bio-inert coating. However, it is not simple to place conventional liquid personal care formulations inside such biodegradable packaging because historically it has been seen that liquid products with high-water content & high-water activity will damage the less persistent biodegradable packaging too quickly to enable a useful shelf life through the existing distribution & supply chains in various markets, even when the biodegradable packaging contains multiple layers including barrier layers. Typically, the following events may occur when placing typical in-market liquid personal care compositions (with high water content & high water activity) inside a biodegradable packaging: a) Early hydrolysis of either the biodegradable sealant polymer i.e., in direct contact with the liquid product or of the biodegradable primer layer causes a significant drop in the polymer molecular weight, making the barrier weaker & more porous; b) The weakened sealant or primer then allows moisture & water to move through it at a higher rate into other layers of the packaging that may be present further into the biodegradable packaging structure. In particular, any metallized barrier layers present typically undergo significant corrosion and damage when this occurs. This corrosion and damage may contribute to even higher moisture & water loss from the package; and/or c) High water content in the formulation led to a high driving force for moisture to leave the package to equilibrate with the outside atmosphere, resulting in high overall weight loss, drying up of the product inside & a significant increase in the product viscosity which eventually makes the product unusable. If any or all of the above occurs, this will lead to a decreased product shelf life that may not be sufficient enough for the typical distribution systems that consumer goods move through, and this can subsequently result in the consumer experiencing poor product performance.

For liquid shampoos specifically, although one solution could be to switch to dry shampoos, we found it could be challenging to move most consumers to dry shampoos immediately (many may never move) due to habit change and these solutions typically require new & expensive capital investments to manufacture the product.

To make any further progress with making biodegradable packages that can hold liquid personal care compositions, the present invention has found that there is a need to understand whether such personal care formulas could in fact be altered sufficiently in order to be less damaging to specific types of biodegradable packaging, whilst the product still delivers its functions.

The present invention is directed to a combination of personal care compositions (with lower water activity and lower water content than the ones in the market today) coupled with specific types of biodegradable packaging having sufficient Moisture Vapor Transmission Rate MVTR barrier to minimize weight loss, whilst also being able to pass specific types of biodegradation test to ensure that it would not be persistent if it escaped into the environment after the package has been used and disposed of. Typically, the present invention has worked to match the water activity of the product to come close to the average humidity in the environment where product is sold, to minimize either weight loss or weight gains. In some cases, work has been done to lower the water activity of the product even further to enable it to be placed within biodegradable packaging with yet worse MVTR barrier, where weight gain is manageable, but weight loss is typically not seen. The solution described enables the marketing of personal care compositions (& potentially other liquid products) that still delight the consumer, inside biodegradable packaging from cellulose fibers that is less persistent than today's alternatives—that still enables a reasonable product shelf life (from 6 months to 2 years) to enable the product to be suitable for consumer usage, even after passing through the typical distribution systems of a typical consumer goods company, from plant to distribution centers to shops to the consumer.

### SUMMARY OF THE INVENTION

The present invention is directed to a biodegradable container in combination with a liquid personal care composition comprising: a biodegradable vessel having at least one opening and a biodegradable cover for the opening wherein the container, a liquid personal care composition comprising from about 14% to about 50% water; from about 20% to about 70% of a humectant; wherein there is a Water Activity (Aw) of about 0.40 to about 0.90.

### BRIEF DESCRIPTION OF THE DRAWINGS

While the specification concludes with claims particularly pointing out and distinctly claiming the subject matter which is regarded as forming the present invention, it is believed that illustrative embodiments of the present invention may be better understood from the following description taken in conjunction with the accompanying drawings, in which:

FIG. 1a is a perspective view of a biodegradable container according to the present disclosure.

FIG. 1b is a perspective view of a biodegradable container including a biodegradable vessel with a fibrous body, having at least one opening and having at least one biodegradable barrier and a flange, filled with a disclosed personal care composition and covered by a biodegradable lid.

FIG. 2 is a perspective view of a biodegradable vessel with at least one opening and having a fibrous body.

FIG. 3 is a cross sectional view of FIG. 1b.

FIG. 4 is a cross sectional view of a pulp molded biodegradable container including a biodegradable vessel with a liquid containment barrier on both the inner and outer surfaces and covered by a biodegradable lid.

FIG. 5 is a cross sectional view of a pulp molded biodegradable container including biodegradable vessel with a liquid containment barrier around all surfaces and covered by a biodegradable lid.

FIG. 6 is a cross sectional view of a biodegradable container including a biodegradable vessel using a biodegradable liquid packaging board and covered by a biodegradable lid.

FIG. 7 is a cross sectional view of a pulp molded biodegradable container including a biodegradable vessel and pulp molded lid with a liquid containment barrier on the inner surface.

FIG. 8 is a cross sectional view of a squeezable biodegradable pulp molded bottle with a liquid containment barrier on the inner surface.

FIG. 9 is a cross sectional view of a squeezable biodegradable pulp molded bottle with a liquid containment barrier covering all surfaces.

FIG. 10 is a cross sectional view of a squeezable biodegradable pulp molded bottle after atomic layer deposition (ALD) of metalized layer.

### DETAILED DESCRIPTION OF EMBODIMENTS OF THE INVENTION

All percentages and ratios used herein are by weight of the total composition, unless otherwise designated. All measurements are understood to be made at ambient conditions, where “ambient conditions” means conditions at about 25° C., under about one atmosphere of pressure, and at about 50% relative humidity, unless otherwise designated. All numeric ranges are inclusive of narrower ranges; delineated upper and lower range limits are combinable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

“Apply” or “application,” as used in reference to a composition, means to apply or spread the compositions of the present invention onto keratinous tissue such as the hair.

“Dermatologically acceptable” means that the compositions or components described are suitable for use in contact with human skin tissue without undue toxicity, incompatibility, instability, allergic response, and the like.

“Safe and effective amount” means an amount of a compound or composition sufficient to significantly induce a positive benefit.

The term “preservation” in the context of the present invention refers to the prevention or retardation of product deterioration due to microorganisms present in the product or composition. A “preservative agent” or “preservative” in the context of the present invention is a substance that prevents or retards the growth of microorganisms in a product or composition.

While the specification concludes with claims particularly pointing out and distinctly claiming the invention, it is believed that the present invention will be better understood from the following description.

As used herein, the term “fluid” includes liquids and gels.

As used herein, the articles including “a” and “an” when used in a claim, are understood to mean one or more of what is claimed or described.

As used herein, “comprising” means that other steps and other ingredients which do not affect the end result can be added. This term encompasses the terms “consisting of” and “consisting essentially of”.

As used herein, “mixtures” is meant to include a simple combination of materials and any compounds that may result from their combination.

As used herein, “molecular weight” or “Molecular weight” refers to the weight average molecular weight unless otherwise stated. Molecular weight is measured using industry standard method, gel permeation chromatography (“GPC”).

Where amount ranges are given, these are to be understood as being the total amount of said ingredient in the composition, or where more than one species fall within the scope of the ingredient definition, the total amount of all ingredients fitting that definition, in the composition. For example, if the composition comprises from 1% to 5% fatty alcohol, then a composition comprising 2% stearyl alcohol and 1% cetyl alcohol and no other fatty alcohol, would fall within this scope.

The amount of each particular ingredient or mixtures thereof described hereinafter can account for up to 100% (or 100%) of the total amount of the ingredient(s) in the personal care composition.

As used herein, “personal care compositions” includes products such as shampoos, shower gels, liquid hand cleansers, hair colorants, facial cleansers, and other surfactant-based liquid compositions

As used herein, the terms “include,” “includes,” and “including,” are meant to be non-limiting and are understood to mean “comprise,” “comprises,” and “comprising,” respectively.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Unless otherwise noted, all component or composition levels are in reference to the active portion of that component or composition, and are exclusive of impurities, for example, residual solvents or by-products, which may be present in commercially available sources of such components or compositions.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein, “Bio-polymers” or “Bio-plastics” are meant to include polymers derived from biological materials, typically plant materials.

As used herein, “Biodegradable” is meant to include materials that are susceptible to being assimilated by microorganisms, such as molds, fungi, and bacteria when the biodegradable material is buried in the ground or otherwise contacts the microorganisms (including contact under environmental conditions conducive to the growth of the microorganisms) and either “readily biodegradable”, “home compostable” or “industrially compostable”. When something is biodegradable it is meant that the entire structure plus all major components pass either one or more of the biodegradation tests listed below. A component is considered a major component if it makes up>10 wt % of the entire structure. If a polymer is regarded as biodegradable after being tested, it is considered to be less persistent than non-biodegradable polymers, in the environment of relevance to the test performed.

As used herein, “bio-inert” refers to inorganic or inorganic-organic hybrid materials that do not interact, respond to, or promote or make worse a chemical reaction or bio activity or other response with any biological materials.

As used herein, “readily biodegradable” or “inherently biodegradable” refers to materials that meet the pass levels for ready biodegradability or inherent biodegradability according to the OECD Guideline for Testing of Chemicals, Method 301 B: CO2 Evolution (Modified Sturm Test) (adopted Jul. 17, 1992).

As used herein, “home compostable” refers to materials that meet the pass levels for the OK compostable HOME OK-02e certification by TÜV AUSTRIA (2012).

As used herein, “industrially compostable” refers to materials that meet the pass levels of the OK industrial compostable certification (EN 13432:2000) by TÜV AUSTRIA (2000).

As used herein, “Copolymer” is meant to include a polymer derived from two or more polymerizable monomers. When used in generic terms the term “copolymer” is also inclusive of more than two distinct monomers, for example, ter-polymers. The term “copolymer” is also inclusive of random copolymers, block copolymers, and graft copolymers.

As used herein, “Cross machine direction” or “CD” is meant to include the width of film, i.e. a direction generally perpendicular to the MD.

As used herein, “Film” is meant to include a sheet-like material wherein the length and width of the material far exceed the thickness of the material. As used herein, the terms “film” and “sheet” are used interchangeably.

As used herein, “Machine direction” or MD is meant to include the length of film as it is produced.

As used herein, “Renewable” is meant to include a material that can be produced or is derivable from a natural source which is periodically (e.g., annually or perennially) replenished through the actions of 15 plants of terrestrial, aquatic or oceanic ecosystems (e.g., agricultural crops, edible and non-edible grasses, forest products, seaweed, or algae), or microorganisms (e.g., bacteria, fungi, or yeast).

As used herein, “recyclable” refers to used paper, including in-plant and post-consumer waste paper and paperboard, which is capable of being processed into new paper or paperboard using the process defined in the Voluntary Standard for Repulping and Recycling Corrugated Fiberboard Treated to Improve its Performance in the Presence of Water and Water Vapor (Aug. 16, 2013).

As used herein, “water-soluble” refers to the ability of a sample material to completely dissolve in or disperse into water leaving no visible solids or forming no visibly separate phase, when at least about 25 grams, at least about 50 grams, at least about 100 grams, at least about 200 grams, of such material is placed in one liter (1 L) of deionized water at 20° C. and under the atmospheric pressure with sufficient stirring.

Conventional biodegradable containers are either not formed exclusively from biodegradable constituents, or they have a comparatively low mechanical stability. The object of the invention is therefore to provide a container which is formed exclusively from biodegradable constituents, which has a high gas tightness and a high mechanical stability when used in combination with liquid personal care composition having lower water activity and lower water content.

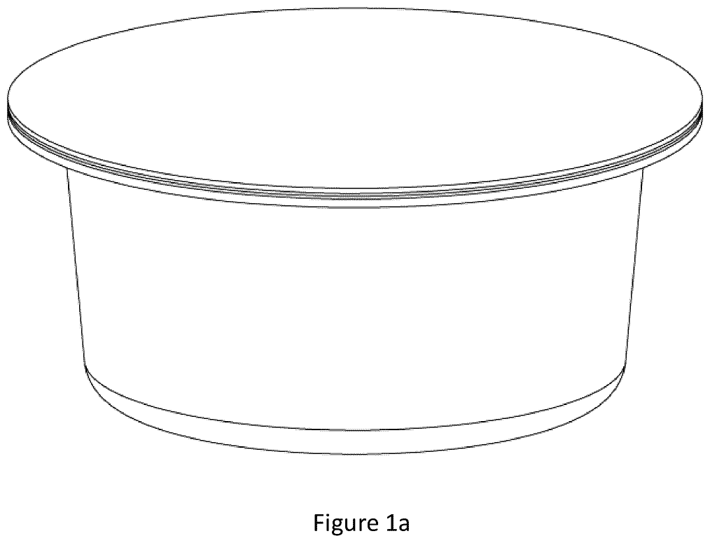

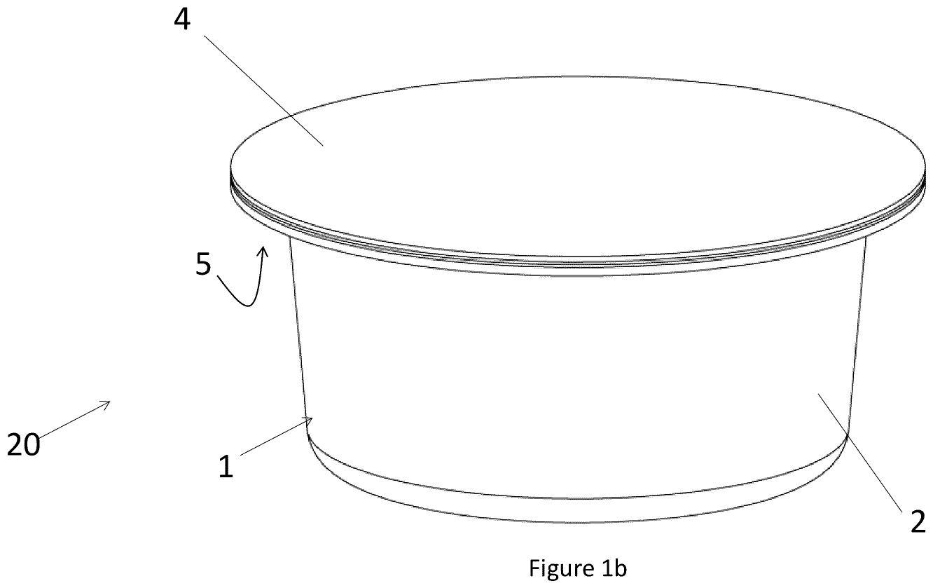

FIG. 1b shows the exemplary biodegradable container 20 with a biodegradable vessel 1 and a biodegradable cover 4. In the present invention, the biodegradable cover 4 can be a biodegradable lidding film. The lidding film is flexible, optimized to deliver the necessary barrier properties such MTVR, OTR and liquid containment as well as biodegradable. The film seals to the container flange 5, thus closing the interior of the container from the environment. The sealing force is optimized to be high enough to both avoid seeping of the liquid content through the seal while being low enough to ensure users can easily peel off the lid to access the product. In the present invention, the peel force can be between 500 to 1000 gf, may be between 700 to 800 gf in a 180° peel test.

The lidding film can have a biodegradable polymeric layer 6 in contact with the product (typically called the sealant or sealant layer or heat sealant layer) that is made from a water in-soluble biodegradable polymer. In some structures, such a biodegradable polymeric layer could also be suitable to act as a lamination layer between other layers such as the paper layer and the sealant layer. The water in-soluble biodegradable polymer can be a thermoplastic polymer. Thermoplastic polymers, as used herein, are polymers that melt and then, upon cooling, crystallize or harden, but can be re-melted upon further heating. Suitable thermoplastic polymers for use herein typically have a melting temperature from 60° C. to 300° C., from 80° C. to 250° C., or from 100° C. to 215° C. The molecular weight of the thermoplastic polymer is sufficiently high to enable entanglement between polymer molecules and yet low enough to be melt extrudable, if needed. Suitable thermoplastic polymers can have weight average molecular weights of 1000 kDa or less, 5 kDa to 800 kDa, 10 kDa to 700 kDa, or 20 kDa to 400 kDa.

The biodegradable water in-soluble polymers can include biodegradable thermoplastic materials selected from the group consisting of aliphatic and/or aromatic polyesters. Such biodegradable aromatic and/or aliphatic polyesters can be either biologically produced (e.g. via large scale bacterial fermentation) or chemically synthesized. Suitable Biodegradable aliphatic and/or aromatic polyester suitable can be a copolymer of: i) at least one aliphatic dicarboxylic acid; and/or ii) at least one aromatic dicarboxylic acid; and iii) a dihydroxy compound (diol). Examples of biodegradable aromatic and/or aliphatic polyesters include, but are not limited to: various co-polyesters of polyethylene terephthalate (PET) and polybutylene terephthalate (PBT) with aliphatic diacids or diols incorporated into the polymer backbone to render such co-polyesters biodegradable or compostable; and various aliphatic polyesters and co-polyesters derived from dibasic acids such as succinic acid, glutaric acid, adipic acid, sebacic acid, azelaic acid, or their derivatives (e.g., alkyl esters, acid chlorides, or their anhydrides), and diols such as ethylene glycol, propylene glycol, 1,4-butanediol, 1,6-hexanediol, 1,4 cyclohexanedimethanol, and the like. For example, the biodegradable aromatic and/or aliphatic polyester may be selected from the group consisting of polybutylene adipate terephthalate (PBAT), polybutylene succinate (PBS), polybutylene succinate adipate (PBSA), polylactic acid (PLA), polycaprolactone (PCL), polyglycolide (PGA), poly propylene carbonate (PPC) and any combinations/mixtures thereof. The aliphatic and/or aromatic polyester used can be from the family of polymers termed as polyhydroxyalkanoates, also known as “PHAs”: these polymers can be synthesized from plant or bacteria fed with a particular substrate, such as glucose, in a fermentation plant. The PHA obtained could be made from a range of various different copolymers. A group of particularly interesting PHA resins are homopolymers and/or copolymers containing repeating structural units of 3-hydroxybutyrate (hereinafter “P3HB”), either alone or in combination with one or more other repeating structural units. The aliphatic and/or aromatic polyester can also include polycaprolactones. The biodegradable water in-soluble polymers can include biodegradable thermoplastic materials selected from the group of thermoplastic starches (e.g., MATER-BI from Novamont or PLANTIC® from Plantic/Kuraray). The starch can be destructured during processing to produce thermoplastic starch composition. The thermoplastic starch composition may also comprise a plasticizer. The biodegradable polymers can be made up of heterogeneous blends of various different biodegradable polymers-since this allows manufacturers to obtain the best balance of properties e.g. balancing biodegradation rates and resistance to the formulations contained within the sealants formed by the biodegradable polymers. One example could include blends of PBAT (e.g. BASF's version marketed under the tradename ECOFLEX®) and PLA (e.g. from Nature Works LLC) which are marketed by BASF under the tradename Ecovio®). PBAT is typically blended with PLA to give more chemical resistance, resistance to hydrolysis or to improve its processability, whilst still balancing the speed of biodegradation and temperature needed for biodegradation to be initiated. Another example of a heterogeneous blend is of PBAT blended with starch which is manufactured by Novamont who market various grades of it under the trade name Materbi. The biodegradable polymeric layers of the present invention may further comprise one or more additives or fillers including, but not limited to, inorganic fillers, alkyd resins, nanoparticles or renewable fillers such as cellulosics (e.g., cotton, wood, hemp, paperboard), lignin, bamboo, straw, grass, kenaf, cellulosic fiber, chitin, chitosan, flax, keratin, algae fillers, natural rubber, nanocrystalline starch, nanocrystalline cellulose, collagen, whey, gluten, and combinations thereof. The biodegradable polymeric layers may further contain one or more other ingredients, such as crystal nucleating agents, lubricants, plasticizers, anti-static agents, flame retardants, conductive additives, heat insulators, cross-linkers, antioxidants, ultraviolet absorbers, colorants, inorganic fillers, organic fillers, hydrolysis inhibitors, lubricants/release agents, extenders, anti-blocking agents, de-tackifying agents and surfactants.

The lidding film can include a cast cellulose layer made by a special film solution casting process. One reason for using such a film is when a very good moisture barrier is required but it is difficult to apply the barrier directly onto the heat sealant layer on the inside of the package, or directly onto the paper layer that might be present on the outside of the package. Instead in such situations, various biodegradable barrier coated cellulose films can be purchased from Futamura under their brand name Natureflex™ and they are placed as an intermediary layer lying between the heat seal layer and any outer layer such as a paper layer. A non-limiting example of such a film is their Natureflex™ NM film which is a metallized cellulose film having a WVTR barrier of approximately 10 g/m2.day at 38° C./90% RH.

The lidding film can include an inorganic barrier layer to promote decreasing the moisture vapor transmission rate (MVTR) of the entire structure. In many cases, the inorganic barrier layer will be laid down from the vapor phase onto the surface of one of the biodegradable polymeric layers present in the structure. In other cases, the inorganic barrier layer may be laid down onto the paper layer. The metalized layer can be transferred to the paper substrate from another substrate that has already been metalized. Suitable vapor-deposited inorganic coatings can be formed of metals including but not limited to aluminum, magnesium, titanium, tin, indium, silicon, carbon, gold, silver, chromium, zinc, copper, cerium, hafnium, tantalum and diamond-like carbon as well as metal oxides (e.g. Al₂O₃ or SiOx), metal nitrides and related compounds. The metalized layer can be transferred to the paper substrate from another substrate that has been already been metalized. The inorganic barrier coating layer can have a thickness of 2-1,000 nm, may be from 10-200 nm, and may be from 20-100 nm. The thickness ratio of the inorganic barrier layer to the polymeric layers may be from about 20 to about 20,000.

The inorganic barrier may include a nano clay layer be laid down from an aqueous nanocomposite dispersion. In the present invention, the inorganic layer laid down from an aqueous coating may be a hectorite clay layer. Additional disclosure for hectorite may be found in U.S. Patent Application Publication No. 2023/0234096 and U.S. Patent Application Publication No. 2023/0235510 incorporated herein by reference. The inorganic layer could also be a Cloisite clay layer as disclosed in the U.S. Patent Application Publication No. 20220112664.

The lidding film can include a biodegradable paper layer also recyclable in typical paper recycling streams. The paper includes mostly cellulose fibers as well as some amounts of polymeric binders, mineral sizing agents, whitening agents, surfactants and other additives. The paper can include also recycled fibers (either natural or synthetic). Non-limiting examples of papers suitable for forming a biodegradable and recyclable paper layer to be part of the present invention include Leine Nature® paper (basis weight=85 g/m2) from Sappi, a machine glazed paper certified “OK Home Compost”; the special kraft paper under brand Lucent from UPM; NiklaSelect V Natural Linen paper (99 g/m²) from Brigl and Bergmeister, a paper sized on one side only; PackPro 7.0 paper (80 g/m²) from Brigl and Bergmeister, a paper sized on both sides; Axello papers from BillerudKorsnäs™ (including from Axello Tough White paper, 80 g/m²) which has been designed to be tougher than many other papers and so which may have some advantages in the distribution chain; and SCG Glassine paper (58 g/m²) from SCG/Prepack. As shown in the TABLE below, these papers pass the paper recycling protocols at both Western Michigan University in the USA and at the PTS Institute in Germany. These papers also pass the OECD 301B biodegradation screening test by undergoing at least 60% biodegradation within 60 days.

| | Western Michigan | PTS Paper | OECD 301B |
|---|---|---|---|
| | Paper | Recycling | Biodegradation |
| Paper Grade | Recycling Protocol | Protocol | Test |
| Leine Nature ® | PASS | PASS | PASS |
| 85 g/m² | | | |
| SappiL | | | |
| NiklaSelect V Natural | PASS | PASS | PASS |
| Linen | | | |
| 100 g/m² | | | |
| Brigl and Bergmeister | | | |
| PackPro 7.0 | PASS | PASS | PASS |
| 80 g/m² | | | |
| Brigl and Bergmeister | | | |
| Axello ® Tough White | PASS | PASS | PASS |
| 80 g/m² | | | |
| BillerudKorsnas | | | |
| Glassine | NA* | NA* | PASS |
| 58 g/m² | | | |
| SCG Packaging | | | |
| Special Kraft Paper | NA* | NA* | NA* |
| 40, 62, 78 and 90 gsm | | | |
| UPM | | | |

*NA-Not Available

The cellulose fibers used to make the paper may be sourced from tree fibers including softwoods and hardwoods and also non-tree fibers which typically have shorter fibers including but not limited to bamboo, grass, hemp, kenaf, flax, corn husks, cotton stalks, coffee grounds, bagasse, rice straw, wheat straw, algae, abaca, sabia grass, esparto grass, milkwood floss fibers, pineapple leaf fibers, wood fibers, pulp fibers and others. Some papers may blend a range of different fibers from different sources.

Indirect Transfer Metallization onto Paper

In the present invention, a metallized layer may not be laid down directly onto a primer layer but instead may be transferred to the paper structure from another substrate that has already been metallized. Sometimes this is called a “Transfer Metallization Process”. This is a process often used in the decoration industry, but there are also some applications where the technique is used in forming barrier layers. In this transfer metallization process, the vacuum metallization layer is firstly deposited onto an intermediate substrate, such as a biaxially oriented PET film, or a biaxially oriented PP film, or a cellulose film etc. to form an intermediate structure and later the metal layer is transferred to the paper-based structure. Potential suppliers of these intermediate structures could include Dongguan Ruize Creative Arts New Materials Co., Ltd or Shanghai Zijiang New Material Technology Co., Ltd or others.

The following describes how these intermediate structures can be formed, as they typically contain multiple layers. Before vacuum metallization, the intermediate substrate is coated with a releasing layer, which will have good adhesion to the metallization layer that will be subsequently laid down on top of it—but with relatively worse adhesion to the underlying intermediate substrate. In some cases this releasing layer may be formed from a Polydimethylsiloxane (PDMS) based material, but other chemistries may also be employed. The vacuum metallization can be done using suitable processes aforementioned to deposit a suitable barrier layer. In some cases—but not always, a final primer layer is deposited on top of the metallization layer to protect it until the intermediate structure is used in a transferring process at some later time or date. Such primer coatings may also later be applied to treat the releasing layer with suitable surface energy/tension so that other coating or lamination layers can be applied to it more easily.

In the transferring process, the vacuum metallized intermediate structure is firstly laminated with the paper substrate onto which there is a need to transfer the vacuum metallization layer. Various suitable adhesives are utilized to carry out this transferring process—and the chosen adhesive is first coated onto the paper substrate. The intermediate structure is then brought in contact with the adhesive coated paper substrate in a lamination process using lamination equipment to form a laminated structure. The adhesive forms higher adhesion between the metallized layer and the paper substrate, than the adhesion between the releasing layer and the intermediate substrate in the intermediate structure. The final part of the lamination process causes the laminated structure to be split into two new structures at the weakest interface (which is now the interface between the releasing layer and the intermediate substrate. The two new structures are the final structure (which will be kept for further processing into packaging) and a disposable structure (which is either disposed of, recycled or reused later several times after thorough cleaning). As such, upon separation of the lamination at the weakest interface of the lamination, the vacuum metallization layer is peeled off the original intermediate structure, together with the releasing layer from the intermediate structure and is transferred onto the paper substrate-thus forming the final structure. This final structure then consists of a paper substrate, an adhesive layer, a vacuum metallized layer and a releasing layer.

Later, this final structure will typically undergo yet another lamination process, in order to adhere an extruded biodegradable sealant layer to the releasing layer side. In some cases, an alternative to laminating an extruded biodegradable sealant layer, is to instead directly coat the biodegradable sealant (in the form of fine polymeric particles) onto the final structure via a process such as emulsion coating or dry coating. At the beginning of this lamination process, typically an anchor coating (sometimes based on polyurethane materials, but other materials could be alternatively used) is coated onto the top surface of the releasing layer in order to modify its surface energy ahead of the biodegradable sealant layer being attached to it, to ensure good adhesion between the releasing layer and the biodegradable sealant layer. One example of such a polyurethane material could be the high functional polyurethane dispersion “TAKELAC™ WPB” series of products from Mitsui Chemicals, for example the TAKELAC WPB-341 grade.

The lidding film can include a biodegradable adhesive to adhere multiple layers together to form a laminate. Nonlimiting examples of the biodegradable adhesive layer can include biodegradable polyvinyl acetates, starches, maltodextrins, natural waxes, artificial waxes and polyester-polyurethane blends. In the present invention, the biodegradable adhesive layer may be a commercially available grade from BASF such as Epotal 3675 or Epotal 3702 or Epotal P100ECO (which is a water-based polyester-polyurethane compostable adhesive or Epotal 3702 (also a water-based adhesive), which are all biodegradable and compostable. In the present invention, the adhesive may be BioTAK® by Berkshire Labels; or Bostik 43298 Thermogrip hotmelt adhesive. In the present invention, the biodegradable adhesive can be water soluble to enhance recyclability in the typical paper repulping system. A non-limiting example of such adhesive includes biodegradable and soluble grades of PVOH and polyethylene oxide.

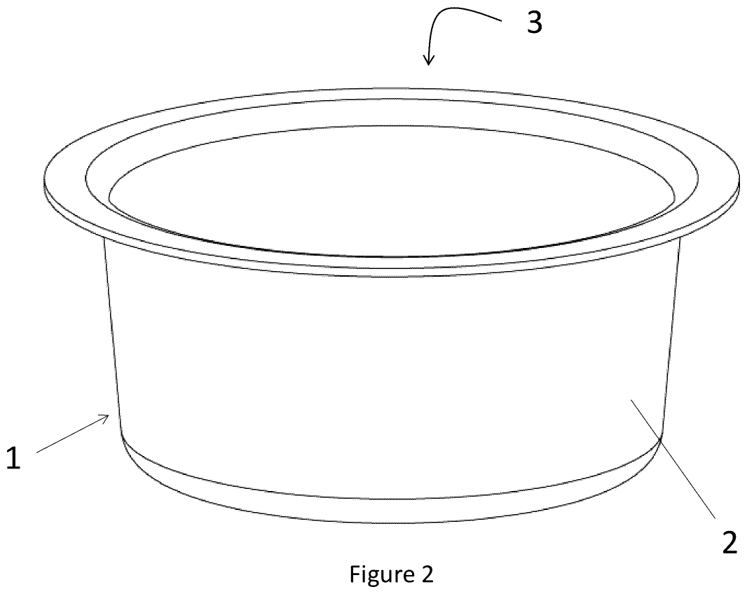

FIG. 2 shows an exemplary biodegradable container 20 including a biodegradable vessel 1 having at least one opening 3.

In the present invention, the biodegradable vessel 1 can be a water in-soluble biodegradable thermoplastic polymer selected from the group consisting of aliphatic and/or aromatic polyesters or thermoplastic starches already mentioned. The biodegradable vessel 1 can be made either using an injection molding or compression molding process.

In the present invention, the biodegradable vessel I may include a fibrous body 2 and at least one biodegradable barrier 7 (not shown).

The fibrous body 2 can be made of an aqueous pulp containing cellulose fibers. This process starts with making a slurry including fibers and additives dispersed in water. In the present text the terms fiber stock, pulp stock and slurry are used synonymously and are fully interchangeable with each other. As used herein “slurry” is a fiber suspension which can consist of 0.5-10% cellulosic fibers and the rest being water and additives. As explained in WO2018/020219, incorporated herein by reference, higher fiber contents affect the suspension's flow characteristics such that it becomes difficult to transport the suspension and to achieve an even coating on the mold. In the present invention, the concentration of fibrous material in the suspending liquid is about 1%. In the wet forming process, the slurry is deposited onto a screened mold to form a layer either by spray coating or, more commonly, by submerging the mold and subsequently applying a vacuum on the rear side of the screen mold. In a second step the slurry layer can be pressed on a tool comprising two mating tool parts, one of which can have a porous wall which contacts the pulp slurry layer and through which vacuum can be drawn to reduce water content. After this pressing step, the molded item is dried out in a heated mold or oven. After the heated mold or oven, the water content can still be about 10-20%. The item can then be subjected to a subsequent press operation applying heat to reduce the surface roughness and porosity and may further reduce the water content to below 10%, may be below 5% and may be below 1%. The average wall thickness of the wet molded part using this process can vary between 0.6 to 1.2 mm, may be from about 0.8 to 1.0 mm. After the pulp molding forming process, protruding edges can be trimmed as required.

Cellulose fibers can be wood or non-wood. Wood fibers can be softwood “long” fibers such as pine, spruce, fir and hemlock or hardwood “short” fibers such as birch, eucalyptus, aspen, acacia and oak. Generally, non-wood plant fibers can be grouped into softwood substitutes such as cotton staple and linters; flax, hemp and kenaf bast fibers; sisal; abaca; bamboo (longer fibers species), and hardwood substitutes such as cereal straws, sugarcane, bagasse, bamboo (shorter fiber species), reeds and grasses, esparto, kenaf (whole stalk or core fiber), corn stalks, sorghum stalks etc. Generally, the fiber recipe is chosen to optimize dewatering and production cycle time, mechanical properties such as burst strength and surface finish (roughness, and porosity). The fibers may include both short and long fibers depending on the desired properties of the final part. The fibers can be extracted using either a bleached or unbleached chemical process, or a mechanical process. The fibers can include recycled fibers.

The slurry can include additives for process control or functionality enhancement. Typical additives for process control include retention aids, anti-foaming agents, Ph-adjustment agents and slime control. Additives for functionality enhancements include (1) fillers such as inorganic mineral fillers; (2) sizing agents such as alkyl ketene dimer (AKD), alkenyl succinic anhydride (ASA), rosin or lignin; (3) additives for dry strength enhancement such as starch, amphoteric, cationic or anionic polyacrylamide resins, enzymes, modified polyamines; (4) additives for wet strength enhancement such as polyamidoamine (PAE)—or polyamine epichlorohydrin, epoxide or cationic glyoxylated resins or (5) micro fibrillated cellulose (MFC) or Cellulose Nano Crystals (CNC) additives. In case the barrier layers are applied by spray or dip coating; the slurry may include some amount of inorganic mineral fillers to close the pores in the surface. In the present invention, the inorganic mineral filler particles may be selected from calcium carbonate as well as platy kaolin or any mixtures thereof.

In the present invention, the slurry may include from 0.5 to 2%, may have about 1% AKD on a dry fiber basis to provide the slurry some waterproofing. In the present invention, an emulsion of Alkenyl succinic anhydride (ASA) or rosin may be used. The slurry can also include less than 0.5% PAE or Glyoxylated Polyacrylamide (GPAM) to provide wet strength to the final article. In the present invention the slurry may include between 2 to 5%, may be between 3 to 4% of MFC on dry fiber basis to improve the surface smoothness for barrier application, stiffness, burst resistance and wet strength. Non-liming Examples of commercially available MFC include CurranO or FiberleanO. This addition is particularly advantageous to improve the barrier effectiveness of a spray or dip coating by decreasing the surface porosity to avoid coating penetration.

In the present invention, the fibrous body 2 may be functionalized after molding by vapor phase deposition of an inorganic barrier layer. As used herein, functionalization is meant to include an alteration of properties of a molded part such as increase hygroscopicity or wet strength, via surface, morphological, and chemical modifications of cellulose fibers. Suitable vapor-deposited inorganic coatings can be formed on pulp fibers from metal or oxides and related compounds. The inorganic barrier layer may be optically opaque, translucent or transparent, depending on the specific chemistry applied. Typically, a metal barrier layer such as aluminum would result in an opaque barrier, whereas a metal oxide barrier such as aluminum oxide or silicon dioxide would results in a transparent barrier.

In the present invention, suitable inorganic coatings may be formed by vapor deposition of metals including but not limited to aluminum, magnesium, titanium, tin, indium, silicon, carbon, gold, silver, chromium, zinc, copper, cerium, hafnium, tantalum and diamond-like carbon. In the present invention, suitable inorganic coatings can be formed by vapor deposition of metal oxides, metal nitrides and related compounds. As used herein, metal oxides include aluminum oxides (e.g. Al₂O₃), aluminum carbide, aluminum nitride, magnesium oxide, titanium oxides (such as titanium dioxide, titanium (3) oxide or titanium monoxide), zinc oxide, tin oxide, yttrium oxide, or zirconium oxides (e.g. zirconium monoxide), calcium oxide, boron oxide or metalloid oxides such as silicon oxides, silicon oxycarbides, and silicon nitrides. Silicon oxide or nitride-based coatings could also be one selected from the group consisting of SiOX (where x is an integer of 1-4) or SiOXNY (where each of x and y is an integer of 1-3). The barrier layer is may be a single component vapor deposition layer comprising at least one selected from the group above, or a dual component vapor deposition layer comprising at least one combination of two components selected from the group consisting of SiOx/Al₂O₃, SiO/ZnO, SiO/CaO, SiO/B2O3 and CaO/Ca(OH) 2. As can be appreciated, metals and metal oxides can be vapor-deposited using a variety of processes. For example, a metal or metal oxide coating may be vapor-deposited using a chemical vapor deposition process or a physical vapor deposition process. Generally, most chemical vapor deposition processes can be suitable due to the stability of the metal, metal oxides and metal oxide precursors. In the present invention, a plasma-assisted chemical vapor deposition process may be used to form the vapor-deposited inorganic coating. In the present invention, an atomic layer chemical vapor deposition process may be used. In the present invention, the inorganic barrier coating layer may have a thickness of 2-1,000 nm, may have a thickness of 10-200 nm, may have a thickness of 20-100 nm. It has been found that this functionalization can significantly increase the wet strength, improve the bulk moisture barrier properties as well as increase the contact angle while preserving the recyclability and remaining bio-inert. This effect is further enhanced when these deposition processes are used in a highly dense pulp matrix and especially in combination with a high refined pulp, MFC's or CNC's. In the present invention, the fibrous body 2 can be functionalized after molding by a parchment treatment to create a naturally hydrophobic surface with high wet strength.

In the present invention, the fibrous body 2 may be molded using a wet pulp mold forming process with fast dewatering and impulse drying as disclosed by Celwise in WO2020/016409 and US 2021/0269983, incorporated herein by reference. This process is found to produce parts with a higher degree of strength and hydrophobicity compared to parts formed by traditional wet forming. It is thought that this may be due to both the fast dewatering enabling cellulose fibers to re-bond with each other quickly as well as high pressure/temperature process enhancing lignin polymerization.

In the present invention, the fibrous body 2 may be molded using a dry molding approach. According to this process the cellulose fibers are transported and formed into the blank using air as a transport medium (“airlay”). Then, the blank is subsequently formed in a press with temperatures above 100° C. and a pressure of at least 1 Mpa. According to this process, additives such as sizing agents can be sprayed or added to the cellulose fibers and/or cellulose blank in solid phase. An example of such process is disclosed by Pulpac in SE541995, SE1851373 and SE543410. Dry molding can be advantageous compared to traditional wet molding by reducing cycle time and energy consumption since there is no need for drying. Parts using this approach are found to be strong but also very flexible. It is hypothesized that this is due to low degree of inter-fiber hydrogen bonds. In the present invention, the pulp molded base may be realized using dry compression molding with a full metal isostatic mold as demonstrated by SACMI to allow a large variety of shapes including ability to mold parts with undercuts while achieving a good degree of dimensional control.

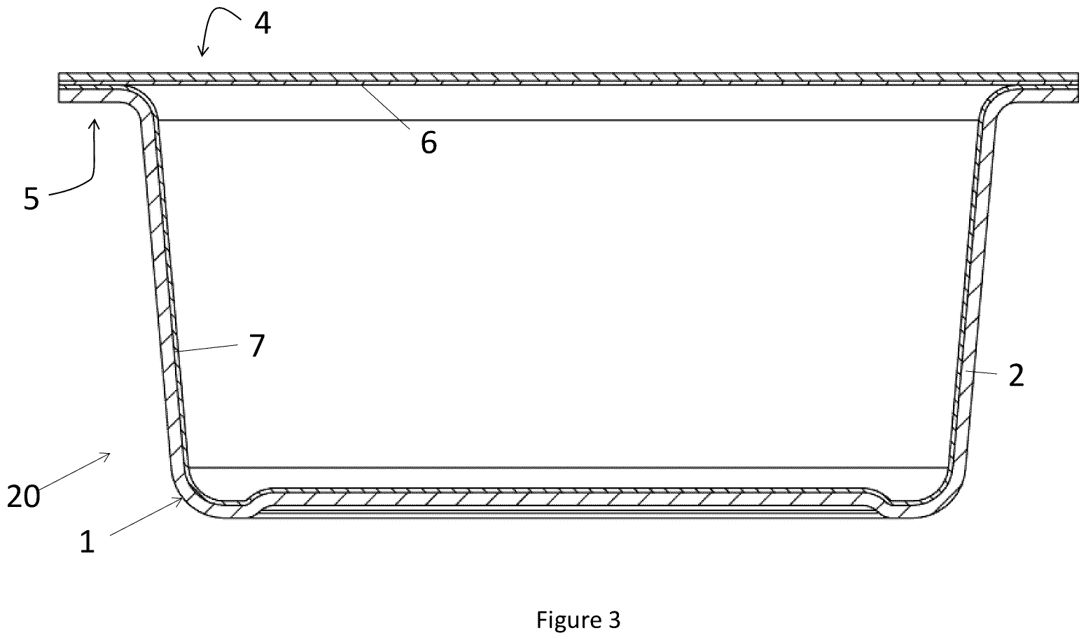

FIG. 3 shows a cross section of the exemplary biodegradable container 20 including a biodegradable vessel and a biodegradable cover 4. As shown in FIG. 3, the vessel includes a liquid containment barrier 7. In the present invention, the liquid containment barrier 7 may be heat sealable to the form an impermeable seal with the lidding film 4 while keeping this still peelable. The liquid containment barrier 7 can be applied to the fibrous body 2 by either spray or may be applied as laminate by welding or gluing.

In the case of spray, the liquid containment barrier 7 can include one of more coatings or layers. In the present invention, the liquid containment barrier can include one or more biodegradable “primer” layers i.e., directly applied to the fibrous body 2, to promote adhesion with subsequent barrier layers and minimize application defects. The primer may be applied in the form of a polymer dispersion, may be applied in the form of an aqueous polymer dispersion, and can include one of the following components: cellulose fibers, polyvinyl alcohol (PVOH), polyvinyl alcohol copolymers, PHA, chitosan, natural gums such as xanthan and carrageenan gum, psyllium husk, sodium alginate, maltodextrin, polysaccharides, casein, whey, agar-agar, certain thermoplastic starch grades and starch can be used as primers. The primer layer can include disintegrants, plasticizers, surfactants, lubricants/release agents, fillers, extenders, anti-blocking agents, detackifying agents, anti-foam, or other functional ingredients. Generally, the thickness of the primer layer should be as thin as possible, but thick enough to form a barrier between the fibrous body 2 and the subsequent layers. The average amount of the primer layer applied on the surface of the molded base may be less than 60 g/m², may be less than 40 g/m², may be less than 20 g/m². After the application of the primer layer, the part can be transferred to a heating unit such as a hot air-drying hood to remove moisture from the coating layer(s) as well as facilitate the film formation by melting or partially melting the polymers in the barrier layers. In the present invention, the drying temperature can be between 100 to 150° C., may be between 110 to 120° C. but other temperatures are admissible depending on the chemistry of the primers. The components of the primer can be dissolved in water and applied as a mixture at the same time. However, the present invention may apply the several priming layers to reduce the incidence of surface defects such as pin-holing, specs or cracks. These different layers can have different composition. The liquid containment barrier layer 7 can include an inorganic barrier layer to promote decreasing the moisture vapor transmission rate (MVTR) of the entire structure. The inorganic barrier layer can be laid down onto the surface of the primer from the vapor phase or using a transfer process. Suitable vapor-deposited inorganic coatings can be formed of metals including but not limited to aluminum, magnesium, titanium, tin, indium, silicon, carbon, gold, silver, chromium, zinc, copper, cerium, hafnium, tantalum and diamond-like carbon as well as metal oxides (e.g. Al₂O₃ or SiOx), metal nitrides and related compounds. The metalized layer can be transferred to the paper substrate from another substrate that has already been metalized. The inorganic barrier coating layer can have a thickness of 2-1,000 nm, may be from 10-200 nm, and may be from 20-100 nm. The thickness ratio of the inorganic barrier layer to the primer layers may be from about 20 to about 20,000. The inorganic barrier can include a nano clay layer be laid down from an aqueous nanocomposite dispersion. In the present invention, the inorganic layer laid down from an aqueous coating may be a hectorite clay layer. Additional disclosure for hectorite may be found in U.S. Patent Application Publication No. 2023/0234096 and U.S. Patent Application Publication No. 2023/0235510 incorporated herein by reference. The inorganic layer could also be a Cloisite clay layer as disclosed in the U.S. Patent Application Publication No. 20220112664 incorporated herein by reference. The liquid containment barrier 7 can include one or more topcoat layers i.e., applied on top of the primer layer to provide additional moisture, oxygen barrier functions, remain integrity in prolonged contact with the product as well as being able to form a peelable seal with the lidding film. The top-coat layer can include linseed oil, carnauba and/or beeswax as taught in WO 2022/258697, incorporated herein by reference. Other waxes meeting the pass level of OECD301B biodegradation screening test can be used. Examples include cutin, rapeseed wax, castor wax, candelilla wax, soy wax, palm oil wax as well as some type of biodegradable paraffin oil-based waxes. The top-coat layer can include also shellac and chitosan. The shellac coating is applied as % ethanol dispersion and dried at 90° C. The topcoat can be an inorganic-organic hybrid-polymer such as bio-ORMOCER® or ORMOCER® developed by The Fraunhofer Institute for Silicate Research in Wurzburg, Germany. These materials are a hybrid between a glass and a polymer, and the exact chemistry of these materials can be tailored to specific applications. Bio-ORMOCER® is modified to be biodegradable. Non-limiting examples of ORMOCER® and bio-ORMOCER® include those described in US. Pat. No. 2011/0250441 A1 and U.S. Pat. No. 6,709,757B2, incorporated herein by reference, in addition to German patents DE-OS 3828098 and DE4303570, incorporated herein by reference. In the present invention, the bio-ORMOCER® topcoat may be dried after application at 120° C. between 2 to 3 minutes to allow complete cross linking. Since the bio-ORMOCER® topcoat is not heat sealable, the vessel 1 is sealed to the lidding film 4 via a biodegradable adhesive aid (not shown). The total surface energy for shellac is found to be 42.2±1.6 mN/m including a dispersion component of 36.6±0.6 mN/m and polar component of 6.6±1.0 mN/m. The total surface energy for chitosan is found to be 48.3±1.2 mN/m including a dispersion component of 33.2±0.6 mN/m and polar component of 15.2±0.6 mN/m. The total surface energy for bio-Ormocer is found to be 46.6±1.2 mN/m including a dispersion component of 36.6±0.8 mN/m and polar component of 10.0±0.4 mN/m. All top-coat chemistries mentioned have been found to be surprisingly stable with the personal care formulations disclosed. Because the thickness of the surface coating affects the recyclability and the biodegradation of the package made from the recyclable barrier paper laminate of the present invention, thinner surface coatings may be used. The amount of each top-coat layer can be less than 26 g/m², may be in the range of 2-26 g/m², may be in the range of 2-5 g/m2.

In the present invention, the liquid containment barrier 7 can be a layer applied by hot vacuum thermoforming. The barrier layer may be made by biodegradable thermoplastic materials selected from the group consisting of aliphatic and/or aromatic polyesters or thermoplastic starches of the groups already mentioned. This may ensure both good formability of the material as well as good adhesion to the lidding film. In the present invention, the laminate includes a binary blend of PLA and PBAT marketed by BASF under the tradename ECOVIOO. The starting thickness of laminate is from 30 to 150 microns, may be between 60 to 90 microns before application depending on the average final thickness targeted. The structure of the film lamination can be optimized and configured based on the performance requirements between barrier properties after application, bonding with pulp surfaces, and recyclability pulp percentage among others. The application process consists in heating the barrier layer to the forming temperature by adhering to a heating plate by a pressure applied by vacuum. The pulp molded cup 2 seats on a mandrel. Once the targeted temperature is achieved, the vacuum on the top plate is released and the barrier layer is draped down by means of vacuum applied on the mandrel side. The bottom mandrel can also be heated to facilitate the adhesion of the laminate to the pulp molded cup. While the liquid containment barrier 7 is shown in FIG. 3 having a uniform wall thickness, in practice a thickness gradient is developed depending on the amount of film stretching during the application process. It is found that applying localized heat to the film i.e., by means of systems such as WATTTRONO can help obtain a more uniform wall thickness and prevent the insurgence of pin holing. The average thickness of the liquid containment barrier 7 after application can be below 90 microns, may be below 75 microns, may be below 50 microns, may be below 20 microns depending on the barrier properties desired.

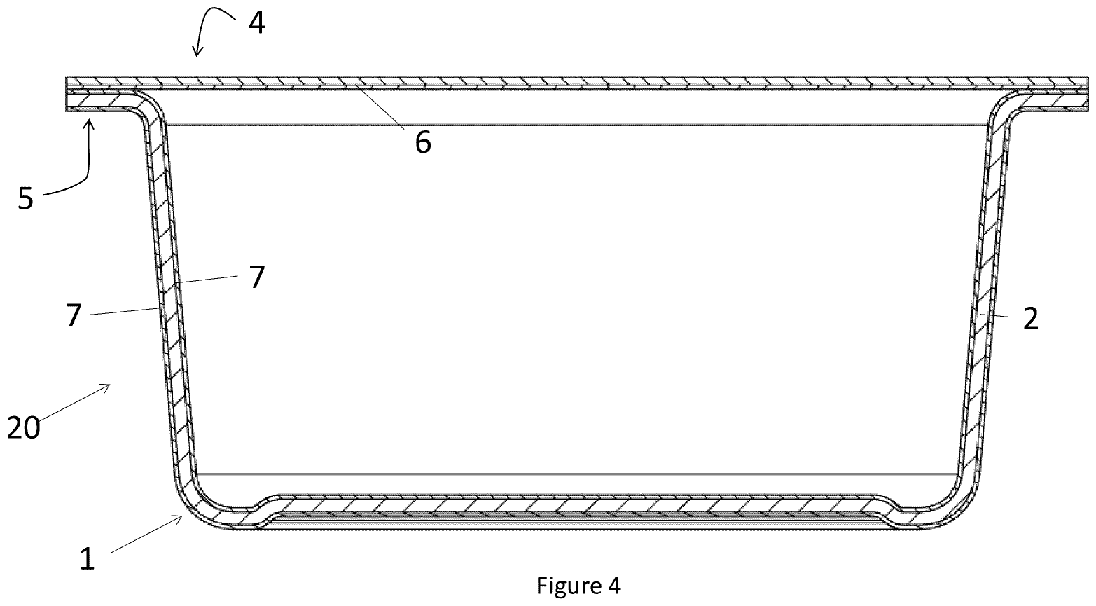

FIG. 4 shows a cross section of the exemplary biodegradable container 20 including a biodegradable vessel 1 with a biodegradable liquid containment barriers applied both on the inner surface as liquid containment barrier 7 as well as outer surface as liquid containment barrier 7. We found that this configuration is particularly useful to boost the barrier properties of the container while retaining its biodegradability.

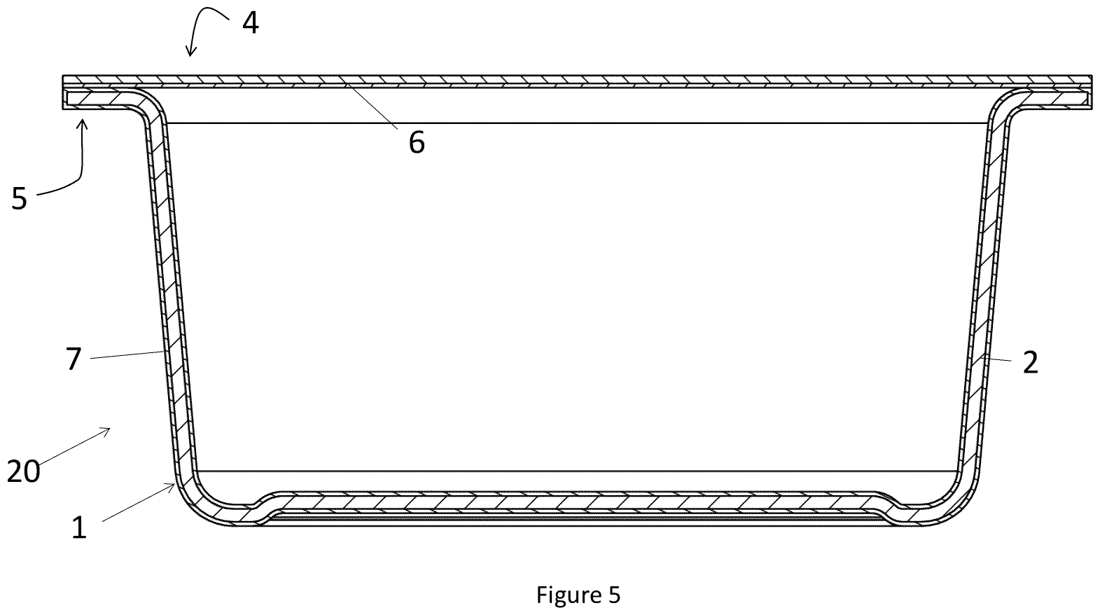

FIG. 5 shows a cross section of the exemplary biodegradable container 20 including a biodegradable vessel 1 and a biodegradable cover, wherein the liquid containment barrier 7 is applied to the outer surface via dip coating. The liquid containment barrier 7 is applied to the outer surface by dip coating and can include one of more coatings or layers. Coatings can include one or more primer layers, an inorganic barrier coating and one or more topcoat layers. The coating can have the same composition described above for spray application. Using this application method, the thickness of each coating layer is typically determined by its viscosity and spin speed. This application method ensures that the container 20 is “sealed” from both the product as well as any external moisture. This has been found to be beneficial to preserve the integrity of the primer layers from moisture stress gradients and ultimately also avoid disruption of the barrier top-coat layers.

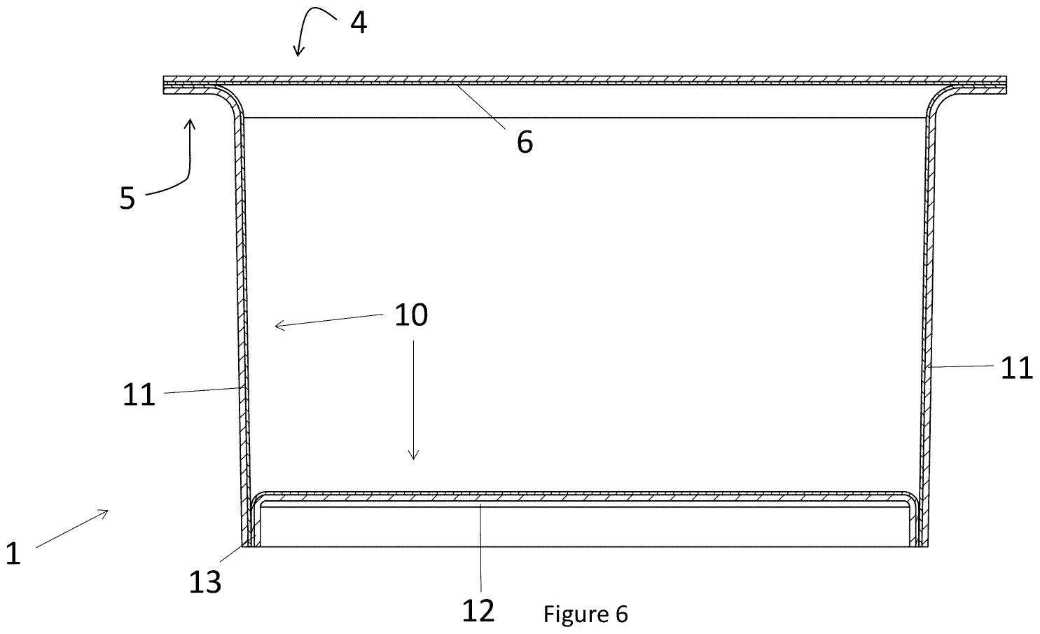

FIG. 6 shows a cross section of the exemplary biodegradable container 20 including a biodegradable vessel 1 and a biodegradable cover, wherein the vessel 1 is made from a biodegradable paper board stock 10. The board stock 10 includes typically a 3-ply paper structure from bleached or unbleached chemical or chemi-thermo-mechanical pulp (CTMP). The board stock 10 is either single or double sided coated with one or more biodegradable thermoplastic materials from the selected from the group consisting of aliphatic and/or aromatic polyesters or thermoplastic starches of the groups already mentioned. Several commercially available solutions exist with such constructions such the Bioware® cup commercialized by Huhtamaki or Cupforma Natura® Bio/2Bio by Stora Enso. While these vessels are typically used for short term storage of liquids such as drinking cups, we have found that these structures are surprisingly stable with the compositions disclosed in this invention. The vessel 1 includes both a formed board side wall 11 as well as a formed base 12 joint together in the seal 13. The side base 12 can formed with a double sited stock. In this case, the side wall 11 can be extended to form another seal with the base 12 (not shown) with a typical cup construction.

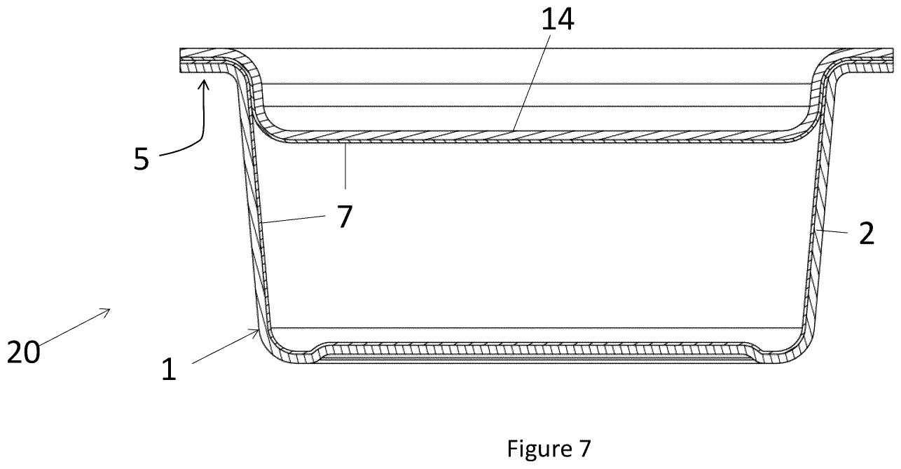

FIG. 7 discloses an exemplary biodegradable container 20 including a biodegradable vessel 1 and a biodegradable cover 14, wherein both the vessel 1 and cover 14 are pulp molded. The vessel 1 and the cover 14 can be two separate pieces or may be as one piece connected by a living hinge. A biodegradable liquid containment barrier 7 can be applied via either spray or dip coating or lamination. The barrier can be applied either on the inside surface of the fibrous body 2 or both inner and outer surfaces. In the present invention, both the vessel 1 and cover 14 may seal by radial interference. An additional biodegradable band (not shown) can be used to make sure the parts don't disassemble during transport such as made by cellulose acetate. In the present invention, the pulp may include fibers and additive to maintain a good elasticity for the proper working of the living hinge. In the present invention, the cellulose fibers forming the pulp molded parts may include softwood, bamboo, and bagasse. The length-weighted average fiber length, the arithmetic average fiber length (ISO 0.2-7.0 mm) and the arithmetic average fiber width according to TAPPI T271 can be determined using a Fiber Image Analyzer such as the Valmet FS5. Softwood fibers can have a length-weighted average fiber length of about 2.25 mm, an arithmetic average fiber length (ISO 0.2-7.0 mm) of about 1.4 mm and an arithmetic average fiber width of about 30 mm. Bamboo fibers can have a length-weighted average fiber length of about 15 mm, an arithmetic average fiber length (ISO 0.2-7.0 mm) of about 1.0 mm and an arithmetic average fiber width of about 15 mm. Bagasse fibers can have a length-weighted average fiber length of about 1.0 mm, an arithmetic average fiber length (ISO 0.2-7.0 mm) of about 0.6 mm and an arithmetic average fiber width of about 22 mm. In the present invention, the fiber count may include between 50 to 60% bamboo, 40 to 50% bagasse and from 0 to 10% softwood.

The slurry of cellulosic fibers can also include additives for process control and/or functionality enhancement. Typical additives for process control include retention aids, anti-foaming agents, Ph-adjustment agents, and slime control. Additives for functionality enhancements include (1) fillers such as inorganic mineral fillers such as calcium carbonate and platy kaolin; (2) sizing agents such as alkyl ketene dimer (AKD), alkenyl succinic anhydride (ASA), rosin or lignin; (3) additives for dry strength enhancement such as starch, amphoteric, cationic or anionic polyacrylamide resins, enzymes, modified polyamines; (4) additives for wet strength enhancement such as polyamidoamine (PAE)—or polyamine epichlorohydrin, epoxide or cationic glyoxylated resins or (5) micro fibrillated cellulose (MFC) or Cellulose Nano Crystals (CNC) additives.

In the present invention, the slurry may include from 0.5 to 2%, may be about 1% AKD on a dry fiber basis to provide the slurry some excellent waterproofing. The slurry can include also between 0.1 to 0.5% PAE to provide to the final article some excellent wet strength. In the present invention, the slurry may include between 2 to 5%, may be between 3 to 4% of MFC on dry fiber basis to improve the surface smoothness for barrier application, stiffness, burst resistance and wet strength. Examples of commercially available MFC include CurranO or Fiberleand. This addition is particularly advantageous to improve the barrier effectiveness of a spray or dip coating by decreasing the surface porosity to avoid coating penetration. In the present invention, the pulp part may be functionalized after molding by a vapor phase deposition of an inorganic barrier layer as previously described.

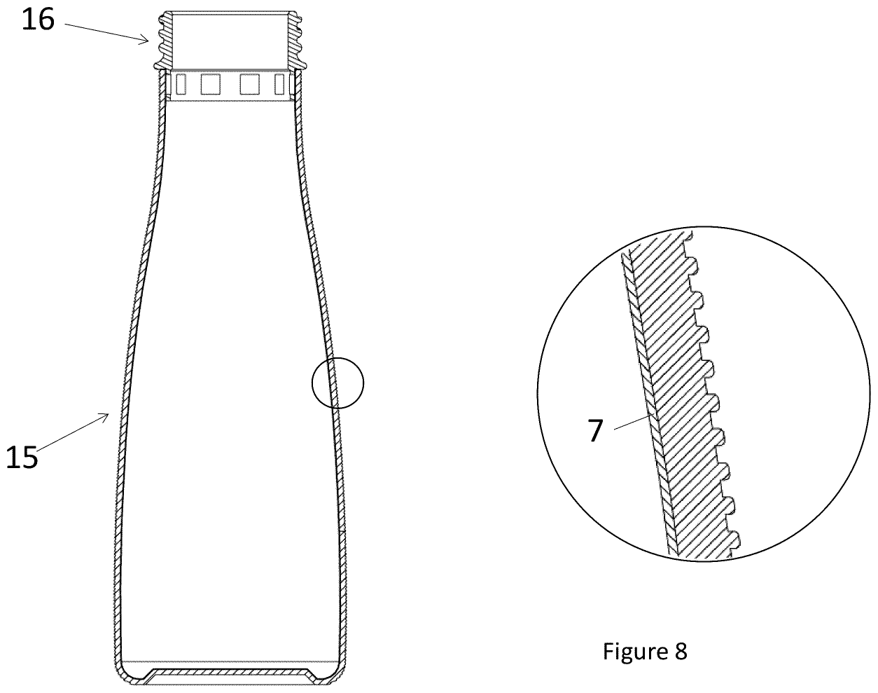

FIG. 8 discloses a cross section of the exemplary biodegradable vessel 1, wherein this is a bottle 15 made of a fibrous cellulose material obtained by wet molding. In the present invention, the bottle may be grafted with an injected thermoplastic starch neck 16 as described in the application WO 2022/258707, incorporated herein by reference. The bottle can engage a biodegradable closure also using thermoplastic starch (not showed). Both the neck and the closure can be coated as described in the application WO 2022/258697, incorporated herein by reference, to protect the components from formula incompatibility. In the present invention, the pulp may include fibers and additive to maintain a good squeezability. In the present invention, the cellulose fibers forming the pulp molded parts may include softwood, bamboo, and bagasse. The length-weighted average fiber length, the arithmetic average fiber length (ISO 0.2-7.0 mm) and the arithmetic average fiber width according to TAPPI T271 can be determined using a Fiber Image Analyzer such as the Valmet FS5. Softwood fibers can have a length-weighted average fiber length of about 2.25 mm, an arithmetic average fiber length (ISO 0.2-7.0 mm) of about 1.4 mm and an arithmetic average fiber width of about 30 mm. Bamboo fibers can have a length-weighted average fiber length of about 15 mm, an arithmetic average fiber length (ISO 0.2-7.0 mm) of about 1.0 mm and an arithmetic average fiber width of about 15 mm. Bagasse fibers can have a length-weighted average fiber length of about 1.0 mm, an arithmetic average fiber length (ISO 0.2-7.0 mm) of about 0.6 mm and an arithmetic average fiber width of about 22 mm. In the present invention, the fiber count may include between 50 to 60% bamboo, 40 to 50% bagasse and from 0 to 10% softwood.

The slurry of cellulosic fibers can also include additives for process control and/or functionality enhancement. Typical additives for process control include retention aids, anti-foaming agents, Ph-adjustment agents, and slime control. Additives for functionality enhancements include (1) fillers such as inorganic mineral fillers such as calcium carbonate and platy kaolin; (2) sizing agents such as alkyl ketene dimer (AKD), alkenyl succinic anhydride (ASA), rosin or lignin; (3) additives for dry strength enhancement such as starch, amphoteric, cationic or anionic polyacrylamide resins, enzymes, modified polyamines; (4) additives for wet strength enhancement such as polyamidoamine (PAE)—or polyamine epichlorohydrin, epoxide or cationic glyoxylated resins or (5) micro fibrillated cellulose (MFC) or Cellulose Nano Crystals (CNC) additives.

In the present invention, the slurry may include from 0.5 to 2%, may be about 1% AKD on a dry fiber basis to provide the slurry some excellent waterproofing. The slurry can include also between 0.1 to 0.5% PAE to provide to the final article some excellent wet strength. In the present invention, the slurry may include between 2 to 5%, may be between 3 to 4% of MFC on dry fiber basis to improve the surface smoothness for barrier application, stiffness, burst resistance and wet strength. Examples of commercially available MFC include CurranO or FiberleanO. This addition is particularly advantageous to improve the barrier effectiveness of a spray or dip coating by decreasing the surface porosity to avoid coating penetration. In the present invention, the pulp part may be functionalized after molding by a vapor phase deposition of an inorganic barrier layer as previously described.

The bottle includes a liquid containment barrier 7 on the inner surface of the bottle 15. The liquid containment barrier 7 on the inner surface the bottle 15 can be applied to the pulp molded bottle by either spray or dip coating. In the case of spray, the liquid containment barrier can include one of more biodegrable coatings or layers including one or more primers as well as one of more top-coats as already previously disclosed.

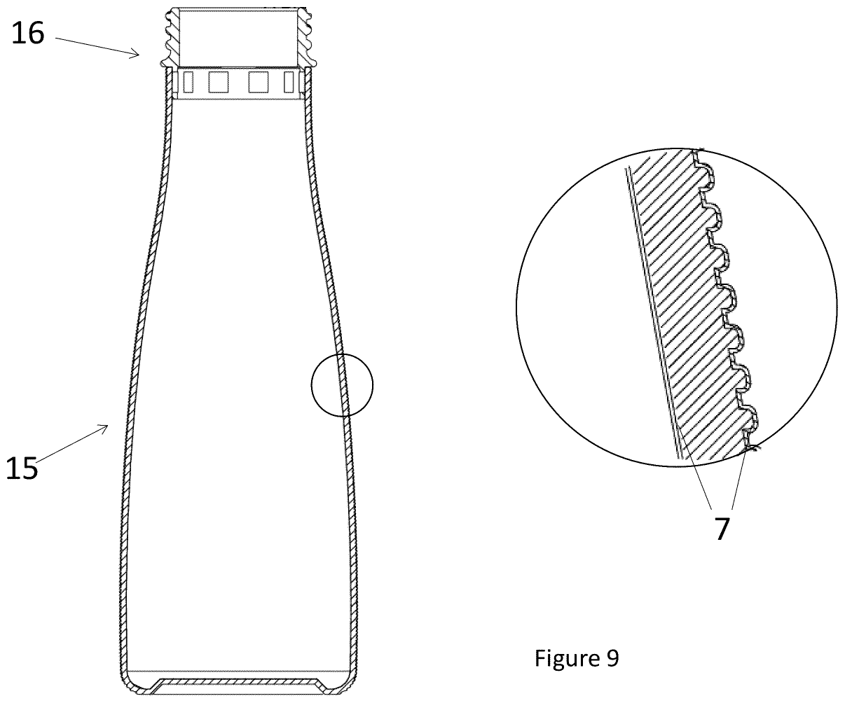

FIG. 9 shows a cross section of the exemplary vessel 1, where in the vessel is a biodegradable bottle 15 and the liquid containment barrier 7 is applied on all component surfaces, the inner and outer surface of biodegradable bottle 15 via dip coating. The liquid containment barrier 7 can include one of more coatings or layers. Coatings can include one or more primer layers, an inorganic barrier coating and one or more topcoat layers. The coating can have the same composition described above for spray application. Using this application method, the thickness of each coating layer is typically determined by its viscosity and spin speed. This application method ensures that the bottle is “sealed” from both the product as well as any external moisture. This has been found to be beneficial to preserve the integrity of the primer layers from moisture stress gradients and ultimately also avoid disruption of the barrier top-coat layers.

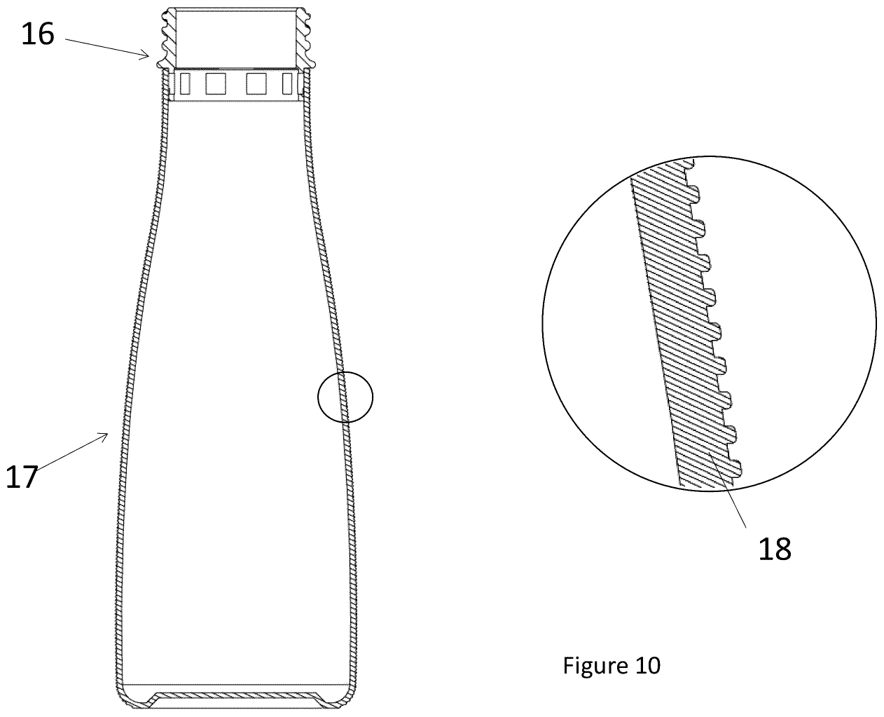

FIG. 10 shows a cross section of the exemplary biodegradable vessel 1, wherein the vessel is a pulp bottle 17, the pulp 18 is primed using MFC and then treated via atomic layer deposition (ALD) after molding to provide the necessary liquid containment properties. PACKAGING TEST METHODS

### 1) Individual Layer Thickness

The thickness of the overall film/individual layers is measured by cutting a 20 μm thick cross-section of a film sample via sliding microtome (e.g. Leica SM2010 R), placing it under an optical microscope in light transmission mode (e.g. Leica Diaplan), and applying an imaging analysis software.

### 2) Caliper

The caliper, or thickness, of a single-layer test sample is measured under a static load by a micrometer, in accordance with compendial method ISO 534, with modifications noted herein. All measurements are performed in a laboratory maintained at 23° C.+2 C.° and 50%+2% relative humidity and test samples are conditioned in this environment for at least 2 hours prior to testing. Caliper is measured with a micrometer equipped with a pressure foot capable of exerting a steady pressure of 70 kPa+0.05 kPa onto the test sample. The micrometer is a dead-weight type instrument with readings accurate to 0.1 micron. A suitable instrument is the TMI Digital Micrometer Model 49-56, available from Testing Machines Inc., New Castle, DE, or equivalent. The pressure foot is a flat ground circular movable face with a diameter that is smaller than the test specimen and capable of exerting the required pressure. A suitable pressure foot has a diameter of 16.0 mm. The test sample is supported by a horizontal flat reference platform that is larger than and parallel to the surface of the pressure foot. The system is calibrated and operated per the manufacturer's instructions. Measurements are made on single-layer test samples taken from rolls or sheets of the raw material, or test samples obtained from a finished package. When excising the test sample from a finished package, use care to not impart any contamination or distortion to the sample during the process. The excised sample should be free from residual adhesive and taken from an area of the package that is free from any seams or folds. The test sample is ideally 200 mm² and must be larger than the pressure foot. To measure caliper, first zero the micrometer against the horizontal flat reference platform. Place the test sample on the platform with the test location centered below the pressure foot. Gently lower the pressure foot with a descent rate of 3.0 mm per second until the full pressure is exerted onto the test sample. Wait 5 seconds and then record the caliper of the test sample to the nearest 0.1 micron. In like fashion, repeat for a total of ten replicate test samples. Calculate the arithmetic mean for all caliper measurements and report the value as Caliper to the nearest 0.1 micron.

### 3) Basis Weight

The basis weight of a test sample is the mass (in grams) per unit area (in square meters) of a single layer of material and is measured in accordance with compendial method ISO 536. The mass of the test sample is cut to a known area, and the mass of the sample is determined using an analytical balance accurate to 0.0001 grams. All measurements are performed in a laboratory maintained at 23° C.+2° C. and 50%+2% relative humidity and test samples are conditioned in this environment for at least 2 hours prior to testing. Measurements are made on test samples taken from rolls or sheets of the raw material, or test samples obtained from a finished package. When excising the test sample from a finished package, use care to not impart any contamination or distortion to the sample during the process. The excised sample should be free from residual adhesive and taken from an area of the package that is free from any seams or folds. The test sample must be as large as possible so that any inherent material variability is accounted for. For flat samples, measure the dimensions of the single layer test sample using a calibrated steel metal ruler traceable to NIST, or equivalent. For non-flat samples, the area can be calculated using 3D data. Calculate the Area of the test sample and record to the nearest 0.0001 square meter. Use an analytical balance to obtain the Mass of the test sample and record to the nearest 0.0001 gram. The weight of a coating can be obtained by subtracting the weight of the coated from the uncoated samples. Calculate Basis Weight by dividing Mass (in grams) by Area (in square meters) and record to the nearest 0.01 grams per square meter (gsm). In like fashion, repeat for a total of ten replicate test samples. Calculate the arithmetic mean for Basis Weight and report to the nearest 0.01 grams/square meter.

### 4) Roughness Measurements (Sq) of Substrate/Individual Layers

Root Mean Square Roughness (Sq) is measured using a 3D Laser Scanning Confocal Microscope such as a Keyence VK-X200 series microscope available from KEYENCE CORPORATION OF AMERICA) which includes a VK-X200K controller and a VK-X210 30 Measuring Unit. The instrument manufacturer's software, VK Viewer version 2.4.1.0, is used for data collection and the manufacturer's software, Multifile Analyzer version 1.1.14.62 and VK Analyzer version 3.4.0.1, are used for data analysis. If needed, the manufacturer's image stitching software, VK Image Stitching version 2.1.0.0, can be used. The manufacturer's analysis software 15377P 22 is compliant with ISO 25178. The light source used is a semiconductor laser with a wavelength of 408 nm and having a power of about 0.95 mW. Heat Seal Strength.Test method ASTM F88-06 can be used to measure the heat seal strength of heat seals formed from the various barrier paper laminates-unless otherwise specified.

### 5) Pinholing Test Method

This is a test method to detect and locate any pin hole equal or greater than 10 μm on a coated surface. The part to test is placed on an absorbent surface with the coated side facing up. Then a dye penetrant solution according to ASTM F3039-23 is spread across the surface under test, may use an eye dropper or pipette and a small roller to apply pressure on the surface to ensure adequate contact. The dye penetrant solution should contact all areas exhibiting questionable surface anomalies taking care not to allow dye penetrant solution to flow over the edge of the sample. Wipe excess dye from sample using a clean absorbent pad and carefully lift the sample. The test is passed is there is no evidence of dye penetration or staining to the opposite side of coated surface.

### 6) Leakage Test Method

This is a test method to measure the ability of a container to prevent leakage when stored or transported.

A minimum of three representative empty vessels and covers of the type being tested are preconditioned for at least 24 hours at 22±3° C., 60%+10 RH. Prepare a tap water solution at room temperature adding a dye such as Rhodamine or Toluidine to give a permanent indication where there is leakage. Fill the specimen at lab ambient temperature with the water/dye solution to expected fill capacity e.g., 150±1 m1, fitted with their respective closure (if applicable) and hermetically closed in the storage configuration. Dry (if needed) any outside surfaces with a (paper) towel so that no product remains on them. Place the specimen in a flat position on a tray capable of holding the liquid should a leak develop. Place some absorbent blotting paper beneath the specimen to detect leakage more easily. Then put the specimen in storage at 25+3° C., 60%+10 RH. No weight or other containers must be placed on top of the specimen being tested. Alternative specimen orientations during the test can be considered such that suspected leak areas are covered with the liquid. Inspect for liquid leakage at 24 h, after 1 week and after 2 weeks. Note location(s) of any eventual leakage. If there is leakage to the outside of the specimen, the package fails the test. If there is no leakage to the outside of the specimen the package passes the test.

### 7) Water Vapor Transmission Rate (WVTR)

This test method is mostly performed according to ASTM F1249-13 under the following test conditions: either the temperature of the test gas is 38° C. (±0.56° C.) and it's relative humidity is 50% (±3%)—or if tropical conditions are required, the temperature of the test gas is set to 38° C. (±0.56° C.) and it's relative humidity to 90% (±3%). The carrier gas is 100% N2 (dry). The equipment used to run the test is a Permatran-W Water Vapor Permeability Instrument following the written procedure QMS 702-004. For materials outside of the Scope (§ 1.1) of ASTM F-1249-13, the water vapor transmission rate test method does not apply. If the barrier properties of a specific substrate are too poor, especially if coatings on paper substrates are very thin and did not enable a good seal to the equipment, then it is not possible to measure the WVTR via ASTM F1249-13. In those cases, a different test method is used i.e., ASTM E96 Cup Test Method. However, results from the two different test methods can still be compared. For ASTM E96, the temperature is 38° C. and the humidity is 90% relative humidity if tropical conditions are desired, or sometimes the humidity is 50% relative humidity if tropical conditions are not needed. For either test method, the water vapor transmission rate is reported in g/m2/day. If normalized by the barrier thickness, the water vapor transmission rate is reported in g.μm/m2/day.

### 8) Oxygen Transmission Rate (OTR)

This test method is mostly performed according to ASTM F1927 under the following test conditions: The temperature of the test gas is 23° C. (±0.56° C.) and it's relative humidity is 80% (±3%) and the test gas concentration is 100% 02, unless otherwise specified. The carrier gas is 98% N2 and 2% H2 and the carrier gas humidity is 0%. Test gas pressure is 760 mmHg. The equipment used to carry out this test is the Oxtran 2/21 Oxygen Permeability Instrument following the test procedure QMS 702-002.For either test method, the oxygen transmission rate is reported in cc/m2/day. If normalized by the barrier thickness, the water vapor transmission rate is reported in cc.μm/m2/day.

### 9) Weight Loss Test Method

This method is used to determine the water weight loss through a container or individual components such as the vessel and cover. A minimum of three representative empty specimens of the type being tested are preconditioned for at least 24 hours at 23±2° C., 60%+10 RH.

Then the specimens are filled with specified amount of tap water or another specified personal care composition at lab ambient temperature to their filled capacity fitted with their respective closure/cover (if applicable) and hermetically closed in the storage configuration. Any different type of closure such as aluminum foil with paraffin should be noted. Dry (if needed) any outer surfaces with a (paper) towel so that no product remains on them.

For flat components, such as the lidding film, the measurement is conduced according to a variant of the ASTM E96 Inverted Cup Water Method. For this test, impermeable cups such as the “vapometer” E96 cups from Thwing-Albert Instrument Co. are filled with 50 g of water or specified personal care composition. The mouth of the cup is 3070 square millimeter in area. The cups are made of noncorroding material, impermeable to water or water vapor. The flat portion of the specimen under measurement is cut into circles slightly larger than the opening of the cup. At least three specimens should be tested representative of the materials and condition being tested. The test specimen is sandwiched between two gaskets and placed on the cup mouth flange assuring the correct orientation. The specimen is then secured to the cup by creating an impermeable seal by tightening an open screw lid.

The weight of the filled covered vessel or cup is recorded with a balance of a resolution of at least 0.01 g. Then specimens are placed in storage at 25+3° C., 60%+10 RH or another relevant testing condition. The specimen should be placed such that the water or the product under test is in direct contact with the specimen being tested. If ASTM E96 cups are used, the cups should be placed in such a way that the air flow is not restricted over the exposed surface. The weight is recorded daily for 2 weeks. The daily weight loss is calculated once the gradient is stabilized at “steady state”. The surface area of the container is calculated. The weight loss is calculated and reported averaging the daily weight loss per a square meter at 25° C., 60% RH or in the relevant tested condition. The test is not applicable if the weight loss doesn't reach a steady state such as in case of a package failure leading to a leak.

### 10) Bottle Squeeze Test Method

This method is used to measure how much force is necessary to dispense a certain amount of product from a bottle. Bottles are filled with Pantene PRO-V Repair & Protect shampoo or another specified personal care composition at the specified filled capacity e.g., 200±1 g and then preconditioned for a minimum of 24 hours at 22±3° C., 60%+10 RH. The bottles are fitted with their respective closure to ensure no leaks.

Each bottle is then placed in a compression tester using a fixture to simulate a squeezing event. An example of compression tester is Z010TN All-round by ZwickRoell GmbH & Co. KG. The load probe has a ¾ inch stainless steel ball attached simulating a thumb pressing on the bottle panel. The bottle is placed horizontally relatively to the load column with the front panel facing up by fixing one bottle extremity at one end resting on two curved aluminum supports (simulating fingers) just about the opposite direction where the load is applied. The bottle is adjusted to ensure the load is applied in the center of the panel and in the middle between the neck (or the bottle base) and the other bottle extremity. Then the probe is lower to contact the bottle reaching a max preload of 0.5 N. A scale with a precision of +0.01 g with a collector plate is placed underneath the package to collect the product dispensed from the orifice during squeezing. The closure is opened making sure no product is leaking from the orifice before the squeeze test. Sometimes it is necessary to re-orient the bottle.

Then the load is applied to the filled bottle at 20 mm/see until a 10 mm displacement is reached. Then, the probe is returned to the start position and another 2 load cycles are performed. The total amount of product dispensed is weighted. A minimum of 3 bottles are tested in total.

Test requirements are met if both the average product collected from each dispensing event from all tested bottles is at least 1 g and all bottles survive the test with no catastrophic failures compromising the bottle functions such as leaking.

### 11) Recyclability in Pulp Stream Based on PTS-RH 021 CAT 2

The testing is carried out with a representative amount of at least 250 g of oven-dry material of the packaging type under test as intended to be disposed by consumers. The first step is to isolate, dry remove and weight non-paper constituents which can be easily separated such as closures, etc. The test material is reduced to specimens of about 2 cm×2 cm and the moisture content determined according to DIN EN ISO 287:2009-09. About 50±1 g of the test material is then disintegrated in a procedure according to DIN EN ISO 5263-1:2004-12. For this purpose, a total volume of 2,000 m1 of specimen is defibrated in a standard disintegrator without prior swelling at a consistency of 2.5%. The disintegration time is 20 minutes, the speed is 3000 rpm, and the temperature of the tap water 40° C. Then, the fiber suspension such obtained is homogenized according to ZM V/6/61. For this purpose, the specimen is transferred into a distributor, diluted with tap water to a form a diluted stock with a consistency of 0.5% and homogenized for about 5 minutes.

Then, the disintegratability is tested after the Zellcheming method ZM V/18/62. For this purpose, the total stock is screened for 5 minutes without any further chemical additive by means of a Brecht-Holl fractionator using a perforated plate with a hole diameter of 0.7 mm. The residue is washed into a 2 liters tank and dewater it through a filter inserted in a Büchner funnel. The filter is folded once and placed in an oven to dry at 105° C. up to weight constancy. Then, the reject is visually inspected and the weighted. To calculate the total reject content, the proportion of removed-dry non-pulp constituents is also included. The fiber yield can be derived from the difference between the (oven-dry, 100%) initial material and the total reject. Products are rated “recyclable” is the total reject does not exceed 20%; “recyclable, but worthy of product design improvement” if the total reject is between 20% to 50%; and “not reasonably usable in paper recycling” if the total reject is above 50% to the initial material input respectively.

For evaluating the undisturbed sheet formation criterion, the total stock is first screened in a procedure after the Zellcheming method ZM V/1.4/86. For this purpose, the total stock is fractionated for 2 minutes by means of a Haindl fractionator using a slot plate of 0.15 mm. The passing fraction, which is hereinafter referred as to ‘accept’ is then collected. Then, the accept is used to form a sheet on a Rapid Köthen sheet former after DIN EN ISO 5269-2:2005-03. Two handsheets of 1.8 g are formed of about 60 gsm. The drying temperature is about 96° C. For the sheet adhesion test, a dried handsheet together with a couch carrier board and a cover sheet are sandwiched between two brass plates and placed in a drying oven where a full surface pressure of 1.18 kPa is applied for 2 minutes. Next, the specimens are placed in an exicator where they are allowed to cool down for 10 minutes, then they undergo the sheet adhesion test and the visual inspection for any optical inhomogeneities.

For the sheet adhesion test, the carrier board and the cover sheet are one by one slowly peeled off the handsheets. While doing so, the test operator checks for potential adhesion effects. Also, the surfaces of the handsheet, cover sheet and carrier board are inspected for any damage or adhesion of the handsheet. The product is considered “recyclable” is no adhesion effect is observed; “limitedly recyclable due to the tackiness in the prepared fiber stock” if some little adhesion effects are observed with slight damage; “not recyclable due to the tackiness in the prepared fiber stock” if adhesion effect with damage is observed.

Then the handsheets are inspected under transmitted light for the presence of any flaws, transparent and white spots, or dirt specks from inks, coating, paint, lamination, and adhesive particles. In addition, the sheets are evaluated for stain from any dark colorants. The product is considered “recyclable” if no or non-disturbing optical inhomogeneities are observed, “limitedly recyclable due to optical inhomogeneities in the prepared fiber stock” if disturbing optical inhomogeneities are observed and “not recyclable due to optical inhomogeneities in the prepared fiber stock” if unacceptable optical inhomogeneities are observed.

### 12) Biodegradation Screening Test OECD 301B

Ideally, the major components of each part and then final package, should all be tested for biodegradation according to the test method OECD 301B.

The final package includes all major and minor (inks, varnishes) components and is open at one end to mimic its disposal after being opened by a consumer. Pass/fail success criteria are shown in TABLE below:

### OECD Biodegradation Test Methods and Pass Criteria

| Test Method | Pass Criteria |
|---|---|
| OECD 301B | 60% thCO₂ evolution |
| Test major components in each layer | or thO₂ consumption |
| Test fully formed sachet* | in 60 days |

The sample should biodegrade at least 60% within 60 days, may be at least 60% within 30 days.

Aerobic biodegradation is measured by the production of carbon dioxide (C02) from the test material in the standard test method as defined by Method 301B test guidelines of the Organization for Economic Cooperation and Development (OECD) the test is run per the indicated OECD test protocols except that it is conducted for 60 days. The polymers may achieve at least 60% of biodegradation as measured by C02 production in 60 days in the standard Method 301B. These OECD test method guidelines are well known in the art and cited herein as a reference {OECD (1992) Test No. 306: Biodegradability in Seawater, OECD Guidelines for the Testing of Chemicals, Section 3, OECD Publishing, Paris, https://doi.org/10.1787/9789264070486-en. and OECD (1992), Test No. 301: Ready Biodegradability, OECD Guidelines for the Testing of Chemicals, Section 3, OECD Publishing, Paris, https://doi.org/10.1787/9789264070349-en.}.

13) OK compost INDUSTRIAL (EN 13432) Test

Packaging or products featuring the OK compost INDUSTRIAL label are guaranteed as biodegradable in an industrial composting plant. This applies to all components, inks and additives. The sole reference point for the certification programme is the harmonized EN 13432:2000 standard: in any event any product featuring the OK compost INDUSTRIAL logo should comply with the requirements of the EU Packaging Directive (94/62/EEC).

One test is a test for disintegration. To pass the disintegration test, the package must disintegrate by 90% within 12 weeks, with any remaining pieces being able to pass through a 2 mm sieve. The temperature must not be raised above 75° C. and after 1 week the temperature must be reduced to 50° C.

This is intended to simulate what would happen inside an actual industrial composting unit.

### 14) OK Compost HOME

Owing to the comparatively smaller volume of waste involved, the temperature in a garden compost heap is clearly lower and less constant than in an industrial composting environment. This is why composting in the garden is a more difficult, slower-paced process. TÜV AUSTRIA's developed OK compost HOME to guarantee complete biodegradability in the light of specific requirements, even in a garden compost heap. OK compost HOME is not based on a standard but is the basis for several standards. It seems important to remember that the OK compost HOME certification program does not explicitly refer to a specific standard but details all the technical requirements that a product must meet in order to obtain the certification. The disintegration test involves ensuring that disintegration occurs within 6 months at a temperature no higher than 30° C. This is intended to simulate what would happen inside an actual home compost.

### 15) Surface Free Energy Calculation

The free surface energy and its dispersion and polar components is estimated by applying a high-speed optical contact angle measuring system. To calculate the free surface energy, contact angles of a series of well-characterized liquids (water, density=1.000 kg/m3; diiodomethane, dens.=3.325 kg/m3; ethylene glycol, dens.=1.113 kg/m3) is used. Static contact angles of liquids on surfaces are evaluated using the sessile drop method (drop volume 2 m1) with circle—or ellipse-fitting. The free surface energy and its components are calculated according to the universal OWRK (Owens-Wendt-Rabel-Kaelble) method. For each coating, 4-5 measurements of the contact angle of each liquid on each surface is conducted. Clean flat glass plate is used as a reference surface (1 sample, 5 measurements per liquid).

### Personal Care Composition

### Detersive Surfactant

The personal care composition may comprise greater than about 1% by weight of a surfactant system which provides cleaning performance to the composition or may comprise greater than 5% by weight of a surfactant system which enables solubilization of a scalp care active and provide a transparent appearance to the composition. In addition, the composition may have sufficient surfactant to enable micellar or polymeric thickening. The surfactant system comprises an anionic surfactant and/or a combination of anionic surfactants and/or a combination of anionic surfactants and co-surfactants selected from the group consisting of amphoteric, zwitterionic, nonionic and mixtures thereof. Various examples and descriptions of detersive surfactants are set forth in U.S. Pat. No. 8,440,605; U.S. Patent Application Publication No. 2009/155383; and U.S. Patent Application Publication No. 2009/0221463, which are incorporated herein by reference in their entirety.

The personal care composition may comprise from about 10% to about 23%, from about 12% to about 21%, from about 10% to about 18%, by weight of one or more surfactants.

Anionic surfactants suitable for use in the compositions are the alkyl and alkyl ether sulfates. Other suitable anionic surfactants are the water-soluble salts of organic, sulfuric acid reaction products. Still other suitable anionic surfactants are the reaction products of fatty acids esterified with isethionic acid and neutralized with sodium hydroxide. Other similar anionic surfactants are described in U.S. Pat. Nos. 2,486,921; 2,486,922; and 2,396,278, which are incorporated herein by reference in their entirety.

Exemplary anionic surfactants for use in the personal care composition include ammonium lauryl sulfate, ammonium laureth sulfate, ammonium C10-15 pareth sulfate, ammonium C10-15 alkyl sulfate, ammonium C11-15 alkyl sulfate, ammonium decyl sulfate, ammonium deceth sulfate, ammonium undecyl sulfate, ammonium undeceth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, sodium C10-15 pareth sulfate, sodium C10-15 alkyl sulfate, sodium C11-15 alkyl sulfate, sodium decyl sulfate, sodium deceth sulfate, sodium undecyl sulfate, sodium undeceth sulfate, potassium lauryl sulfate, potassium laureth sulfate, potassium C10-15 pareth sulfate, potassium C10-15 alkyl sulfate, potassium C11-15 alkyl sulfate, potassium decyl sulfate, potassium deceth sulfate, potassium undecyl sulfate, potassium undeceth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. The anionic surfactant may be sodium lauryl sulfate or sodium laureth sulfate.

The composition of the present invention can also include anionic surfactants selected from the group consisting of:
a) R₁O(CH₂CHR₃O)_{y}SO₃M;
b) CH₃(CH₂)_{z}CHR₂CH₂ O (CH₂ CHR₃O)_{y}SO₃M; and
c) mixtures thereof,

where R₁ represents CH₃ (CH₂) 10, R₂ represents H or a hydrocarbon radical comprising 1 to 4 carbon atoms such that the sum of the carbon atoms in z and R₂ is 8, R₃ is H or CH₃, y is 0 to 7, the average value of y is about 1 when y is not zero (0), and M is a monovalent or divalent, positively-charged cation.

Suitable anionic alkyl sulfates and alkyl ether sulfate surfactants include, but are not limited to, those having branched alkyl chains which are synthesized from C8 to C18 branched alcohols which may be selected from the group consisting of: Guerbet alcohols, aldol condensation derived alcohols, oxo alcohols, F-T oxo alcohols and mixtures thereof. Non-limiting examples of the 2-alkyl branched alcohols include oxo alcohols such as 2-methyl-1-undecanol, 2-ethyl-1-decanol, 2-propyl-1-nonanol, 2-butyl 1-octanol, 2-methyl-1-dodecanol, 2-ethyl-1-undecanol, 2-propyl-1-decanol, 2-butyl-1-nonanol, 2-pentyl-1-octanol, 2-pentyl-1-heptanol, and those sold under the tradenames LIAL® (Sasol), ISALCHEM® (Sasol), and NEODOL® (Shell), and Guerbet and aldol condensation derived alcohols such as 2-ethyl-1-hexanol, 2-propyl-1-butanol, 2-butyl-1-octanol, 2-butyl-1-decanol, 2-pentyl-1-nonanol, 2-hexyl-1-octanol, 2-hexyl-1-decanol and those sold under the tradename ISOFOL® (Sasol) or sold as alcohol ethoxylates and alkoxylates under the tradenames LUTENSOL XP® (BASF) and LUTENSOL XL® (BASF).

The anionic alkyl sulfates and alkyl ether sulfates may also include those synthesized from C8 to C18 branched alcohols derived from butylene or propylene which are sold under the trade names EXXAL™ (Exxon) and Marlipal® (Sasol). This includes anionic surfactants of the subclass of sodium trideceth-n sulfates (STnS), where n is between about 0.5 and about 3.5. Exemplary surfactants of this subclass are sodium trideceth-2 sulfate and sodium trideceth-3 sulfate. The composition of the present invention can also include sodium tridecyl sulfate.

The composition of the present invention can also include anionic alkyl and alkyl ether sulfosuccinates and/or dialkyl and dialkyl ether sulfosuccinates and mixtures thereof. The dialkyl and dialkyl ether sulfosuccinates may be a C6-15 linear or branched dialkyl or dialkyl ether sulfosuccinate. The alkyl moieties may be symmetrical (i.e., the same alkyl moieties) or asymmetrical (i.e., different alkyl moieties). Nonlimiting examples include: disodium lauryl sulfosuccinate, disodium laureth sulfosuccinate, sodium bistridecyl sulfosuccinate, sodium dioctyl sulfosuccinate, sodium dihexyl sulfosuccinate, sodium dicyclohexyl sulfosuccinate, sodium diamyl sulfosuccinate, sodium diisobutyl sulfosuccinate, linear bis(tridecyl) sulfosuccinate and mixtures thereof.

The personal care composition may comprise a co-surfactant. The co-surfactant can be selected from the group consisting of amphoteric surfactant, zwitterionic surfactant, non-ionic surfactant and mixtures thereof. The co-surfactant can include, but is not limited to, lauramidopropyl betaine, cocoamidopropyl betaine, lauryl hydroxysultaine, sodium lauroamphoacetate, disodium cocoamphodiacetate, cocamide monoethanolamide and mixtures thereof.

The personal care composition may further comprise from about 0.5% to about 8%, from about 1.0% to about 7%, from about 1.5% to about 6%, by weight of one or more amphoteric, zwitterionic, nonionic co-surfactants, or a mixture thereof.

Suitable amphoteric or zwitterionic surfactants for use in the personal care composition herein include those which are known for use in shampoo or other personal care cleansing. Non limiting examples of suitable zwitterionic or amphoteric surfactants are described in U.S. Pat. Nos. 5,104,646 and 5,106,609, which are incorporated herein by reference in their entirety.

Amphoteric co-surfactants suitable for use in the composition include those surfactants described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Suitable amphoteric surfactant include, but are not limited to, thoseselected from the group consisting of: sodium cocaminopropionate, sodium cocaminodipropionate, sodium cocoamphoacetate, sodium cocoamphodiacetate, sodium cocoamphohydroxypropylsulfonate, sodium cocoamphopropionate, sodium cornamphopropionate, sodium lauraminopropionate, sodium lauroamphoacetate, sodium lauroamphodiacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphodiacetate, ammonium cocoamphohydroxypropylsulfonate, ammonium cocoamphopropionate, ammonium cornamphopropionate, ammonium lauraminopropionate, ammonium lauroamphoacetate, ammonium lauroamphodiacetate, ammonium lauroamphohydroxypropylsulfonate, ammonium lauroamphopropionate, ammonium cornamphopropionate, ammonium lauriminodipropionate, triethanolamine cocaminopropionate, triethanolamine cocaminodipropionate, triethanolamine cocoamphoacetate, triethanolamine cocoamphohydroxypropylsulfonate, triethanolamine cocoamphopropionate, triethanolamine cornamphopropionate, triethanolamine lauraminopropionate, triethanolamine lauroamphoacetate, triethanolamine lauroamphohydroxypropylsulfonate, triethanolamine lauroamphopropionate, triethanolamine cornamphopropionate, triethanolamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethyl-7 carboxyamphodiacetate, lauraminopropionic acid, lauroamphodipropionic acid, lauryl aminopropylglycine, lauryl diethylenediaminoglycine, and mixtures thereof

The composition may comprises a zwitterionic co-surfactant, wherein the zwitterionic surfactant is a derivative of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. The zwitterionic surfactant can be selected from the group consisting of: cocamidoethyl betaine, cocamidopropylamine oxide, cocamidopropyl betaine, cocamidopropyl dimethylaminohydroxypropyl hydrolyzed collagen, cocamidopropyldimonium hydroxypropyl hydrolyzed collagen, cocamidopropyl hydroxysultaine, cocobetaineamido amphopropionate, coco-betaine, coco-hydroxysultaine, coco/oleamidopropyl betaine, coco-sultaine, lauramidopropyl betaine, lauryl betaine, lauryl hydroxysultaine, lauryl sultaine, and mixtures thereof.

Suitable nonionic surfactants for use in the present invention include those described in McCutcheion's Detergents and Emulsifiers, North American edition (1986), Allured Publishing Corp., and McCutcheion's Functional Materials, North American edition (1992). Suitable nonionic surfactants for use in the personal care compositions of the present invention include, but are not limited to, polyoxyethylenated alkyl phenols, polyoxyethylenated alcohols, polyoxyethylenated polyoxypropylene glycols, glyceryl esters of alkanoic acids, polyglyceryl esters of alkanoic acids, propylene glycol esters of alkanoic acids, sorbitol esters of alkanoic acids, polyoxyethylenated sorbitor esters of alkanoic acids, polyoxyethylene glycol esters of alkanoic acids, polyoxyethylenated alkanoic acids, alkanolamides, N-alkylpyrrolidones, alkyl glycosides, alkyl polyglucosides, alkylamine oxides, and polyoxyethylenated silicones.

The co-surfactant can be a non-ionic surfactant selected from the alkanolamides group including: Cocamide, Cocamide Methyl MEA, Cocamide DEA, Cocamide MEA, Cocamide MIPA, Lauramide DEA, Lauramide MEA, Lauramide MIPA, Myristamide DEA, Myristamide MEA, PEG-20 Cocamide MEA, PEG-2 Cocamide, PEG-3 Cocamide, PEG-4 Cocamide, PEG-5 Cocamide, PEG-6 Cocamide, PEG-7 Cocamide, PEG-3 Lauramide, PEG-5 Lauramide, PEG-3 Oleamide, PPG-2 Cocamide, PPG-2 Hydroxyethyl Cocamide, PPG-2 Hydroxyethyl Isostearamide and mixtures thereof.

Representative polyoxyethylenated alcohols include alkyl chains ranging in the C9-C16 range and having from about 1 to about 110 alkoxy groups including, but not limited to, laureth-3, laureth-23, ceteth-10, steareth-10, steareth-100, beheneth-10, and commercially available from Shell Chemicals, Houston, Texas under the trade names Neodol® 91, Neodol® 23, Neodol® 25, Neodol® 45, Neodol® 135, Neodo®1 67, Neodol® PC 100, Neodol® PC 200, Neodol® PC 600, and mixtures thereof.

Also available commercially are the polyoxyethylene fatty ethers available commercially under the Brij® trade name from Uniqema, Wilmington, Delaware, including, but not limited to, Brij® 30, Brij® 35, Brij® 52, Brij® 56, Brij® 58, Brij® 72, Brij® 76, Brij® 78, Brij® 93, Brij® 97, Brij® 98, Brij® 721 and mixtures thereof.

Suitable alkyl glycosides and alkyl polyglucosides can be represented by the formula(S) n-O-R wherein S is a sugar moiety such as glucose, fructose, mannose, galactose, and the like; n is an integer of from about 1 to about 1000, and R is a C8-C30 alkyl group. Examples of long chain alcohols from which the alkyl group can be derived include decyl alcohol, lauryl alcohol, myristyl alcohol, cetyl alcohol, stearyl alcohol, oleyl alcohol, and the like. Examples of these surfactants include alkyl polyglucosides wherein S is a glucose moiety, R is a C8-20 alkyl group, and n is an integer of from about 1 to about 9. Commercially available examples of these surfactants include decyl polyglucoside and lauryl polyglucoside available under trade names APG® 325 CS, APG® 600 CS and APG® 625 CS) from Cognis, Ambler, Pa. Also useful herein are sucrose ester surfactants such as sucrose cocoate and sucrose laurate and alkyl polyglucosides available under trade names Triton™ BG-10 and Triton™ CG-110 from The Dow Chemical Company, Houston, Tx.

Other nonionic surfactants suitable for use in the present invention are glyceryl esters and polyglyceryl esters, including but not limited to, glyceryl monoesters, glyceryl monoesters of C12-22 saturated, unsaturated and branched chain fatty acids such as glyceryl oleate, glyceryl monostearate, glyceryl monopalmitate, glyceryl monobehenate, and mixtures thereof, and polyglyceryl esters of C12-22 saturated, unsaturated and branched chain fatty acids, such as polyglyceryl-4 isostearate, polyglyceryl-3 oleate, polyglyceryl-2-sesquioleate, triglyceryl diisostearate, diglyceryl monooleate, tetraglyceryl monooleate, and mixtures thereof.

Also useful herein as nonionic surfactants are sorbitan esters. Sorbitan esters of C12-22 saturated, unsaturated, and branched chain fatty acids are useful herein. These sorbitan esters usually comprise mixtures of mono-, di-, tri-, etc. esters. Representative examples of suitable sorbitan esters include sorbitan monolaurate (SPAN® 20), sorbitan monopalmitate (SPAN® 40), sorbitan monostearate (SPAN® 60), sorbitan tristearate (SPAN® 65), sorbitan monooleate (SPAN® 80), sorbitan trioleate (SPAN® 85), and sorbitan isostearate.

Also suitable for use herein are alkoxylated derivatives of sorbitan esters including, but not limited to, polyoxyethylene (20) sorbitan monolaurate (Tween® 20), polyoxyethylene (20) sorbitan monopalmitate (Tween® 40), polyoxyethylene (20) sorbitan monostearate (Tween® 60), polyoxyethylene (20) sorbitan monooleate (Tween® 80), polyoxyethylene (4) sorbitan monolaurate (Tween® 21), polyoxyethylene (4) sorbitan monostearate (Tween® 61), polyoxyethylene (5) sorbitan monooleate (Tween® 81), and mixtures thereof, all available from Uniqema.

Also suitable for use herein are alkylphenol ethoxylates including, but not limited to, nonylphenol ethoxylates (Tergitol™ NP-4, NP-6, NP-7, NP-8, NP-9, NP-10, NP-11, NP-12, NP-13, NP-15, NP-30, NP-40, NP-50, NP-55, NP-70 available from The Dow Chemical Company, Houston, Tx.) and octylphenol ethoxylates (Triton™ X-15, X-35, X-45, X-114, X-100, X-102, X-165, X-305, X-405, X-705 available from The Dow Chemical Company, Houston, TX).

Also suitable for use herein are tertiary alkylamine oxides including lauramine oxide and cocamine oxide.

Non limiting examples of other anionic, zwitterionic, amphoteric, and non-ionic additional surfactants suitable for use in the personal care composition are described in Mccutcheon's, Emulsifiers and Detergents, 1989 Annual, published by M. C. Publishing Co., and U.S. Pat. Nos. 3,929,678, 2,658,072; 2,438,091; 2,528,378, which are incorporated herein by reference in their entirety.

Suitable surfactant combinations comprise an average weight % of alkyl branching of from about 0.5% to about 30%, from about 1% to about 25%, from about 2% to about 20%. The surfactant combination can have a cumulative average weight % of C8 to C12 alkyl chain lengths of from about 7.5% to about 25%, from about 10% to about 22.5%, from about 10% to about 20%. The surfactant combination can have an average C8-C12/C13-C18 alkyl chain ratio from about 3 to about 200, from about 25 to about 175.5, from about 50 to about 150, from about 75 to about 125.

### Humectant

The present invention may comprise a humectant. Humectants have an affinity to hydrogen bonds of water molecules. Non-limiting examples of suitable humectants for use in the present invention may include the following: amino acids and derivatives thereof such as proline and arginine aspartate, 1,3-butylene glycol, propylene glycol and water and codium tomentosum extract, collagen amino acids or peptides, creatinine, diglycerol, biosaccharide gum-1, glucamine salts, glucuronic acid salts, glutamic acid salts, polyethylene glycol ethers of glycerine (e. g. glycereth 20), glycerine, glycerol monopropoxylate, glycogen, hexylene glycol, honey, and extracts or derivatives thereof, hydrogenated starch hydrolysates, hydrolyzed mucopolysaccharides, inositol, keratin amino acids, LAREX A-200 (available from Larex), glycosaminoglycans, methoxy PEG 10, methyl gluceth-10 and −20 (both commercially available from Amerchol located in Edison, N.J.), methyl glucose, 3-methyl-1,3-butanediol, N-acetyl glucosamine salts, polyethylene glycol and derivatives thereof (such as PEG 15 butanediol, PEG 4, PEG 5 pentaerythitol, PEG 6, PEG 8, PEG 9), pentaerythitol, 1,2 pentanediol, PPG-1 glyceryl ether, PPG-9,2-pyrrolidone-5-carboxylic acid and its salts such as glyceryl pca, saccharide isomerate, SEACARE (available from Secma), sericin, silk amino acids, sodium acetylhyaluronate, sodium hyaluronate, sodium poly-aspartate, sodium polyglutamate, sorbeth 20, sorbeth 6, sugar and sugar alcohols and derivatives thereof such as glucose, sucrose, fructose, mannose and polyglycerol sorbitol, trehalose, triglycerol, trimethyolpropane, tris(hydroxymethyl) amino methane salts, and yeast extract, and mixtures thereof, ionic salts such as sodium chloride and potassium chloride and mixtures thereof.

In the present invention, the humectants may be polyhydric alcohols selected from the group consisting of glycerin, diglycerin, glycerol, erythritol, arabitol, xylitol, ribitol, mannitol, sorbitol, galactitol, fucitol, maltitol, mannose, inositol, triethyleneglycol, sodium pyrrolidone carboxylic acid (PCA), zinc PCA and derivatives and mixtures thereof.

The composition contains a safe and effective amount of the humectant. In particular, it may contain from about 20% to about 70%; from about 20% to about 50%; from about 23% to about 45%; by weight of a humectant.

In the present invention, the composition may contain two or more different humectants, for example, the composition may contain glycerin and xylitol.

### Thickening Polymers

The personal care composition may comprise a thickening polymer to increase the viscosity of the composition. Suitable thickening polymers can be used. The personal care composition may comprise from about 0.05% to about 10% of a thickening polymer, from about 0.05% to about 5% of a thickening polymer, from about 0.05% to about 2.5% % of a thickening polymer, and from about 0.05% % to about 2% of a thickening polymer. The thickening polymer modifier may be a polyacrylate, polyacrylamide thickeners. The thickening polymer may be an anionic thickening polymer.

The personal care composition may comprise thickening polymers that are homopolymers based on acrylic acid, methacrylic acid or other related derivatives, non-limiting examples include polyacrylate, polymethacrylate, polyethylacrylate, and polyacrylamide.

The thickening polymers may be alkali swellable and hydrophobically-modified alkali swellable acrylic copolymers or methacrylate copolymers, non-limiting examples include acrylic acid/acrylonitrogens copolymer, acrylates/steareth-20 itaconate copolymer, acrylates/ceteth-20 itaconate copolymer, Acrylates/Aminoacrylates/C10-30 Alkyl PEG-20 Itaconate Copolymer, acrylates/aminoacrylates copolymer, acrylates/steareth-20 methacrylate copolymer, acrylates/beheneth-25 methacrylate copolymer, acrylates/steareth-20 methacrylate crosspolymer, acrylates/beheneth-25 methacrylate/HEMA crosspolymer, acrylates/vinyl neodecanoate crosspolymer, acrylates/vinyl isodecanoate crosspolymer, Acrylates/Palmeth-25 Acrylate Copolymer, Acrylic Acid/Acrylamidomethyl Propane Sulfonic Acid Copolymer, and acrylates/C10-C30 alkyl acrylate crosspolymer.

The thickening polymers may be soluble crosslinked acrylic polymers, a non-limiting example includes carbomers.

The thickening polymers may be an associative polymeric thickeners, non-limiting examples include: hydrophobically modified, alkali swellable emulsions, non-limiting examples include hydrophobically modified polypolyacrylates; hydrophobically modified polyacrylic acids, and hydrophobically modified polyacrylamides; hydrophobically modified polyethers wherein these materials may have a hydrophobe that can be selected from cetyl, stearyl, oleayl, and combinations thereof.

The thickening polymers may be used in combination with polyvinylpyrrolidone, crosslinked polyvinylpyrrolidone and derivatives. The thickening polymers may be combined with polyvinyalcohol and derivatives. The thickening polymers may be combined with polyethyleneimine and derivatives.

The thickening polymers may be combined with alginic acid based materials, non-limiting examples include sodium alginate, and alginic acid propylene glycol esters.

The thickening polymers may be used in combination with polyurethane polymers, non-limiting examples include: hydrophobically modified alkoxylated urethane polymers, non-limiting examples include PEG-150/decyl alcohol/SMDI copolymer, PEG-150/stearyl alcohol/SMDI copolymer, polyurethane-39.

The thickening polymers may be combined with an associative polymeric thickeners, non-limiting examples include: hydrophobically modified cellulose derivatives; and a hydrophilic portion of repeating ethylene oxide groups with repeat units from about 10 to about 300, from about 30 to about 200, from about 40 to about 150. Non-limiting examples of this class include PEG-120-methylglucose dioleate, PEG-(40 or 60) sorbitan tetraoleate, PEG-150 pentaerythrityl tetrastearate, PEG-55 propylene glycol oleate, PEG-150 distearate.

The thickening polymers may be combined with cellulose and derivatives, non-limiting examples include microcrystalline cellulose, carboxymethylcelluloses, hydroxyethylcellulose, hydroxypropylcellulose, hydroxypropyl methylcellulose, methylcellulose, ethyl cellulose; nitro cellulose; cellulose sulfate; cellulose powder; hydrophobically modified celluloses.

The thickening polymers may be combined with a guar and guar derivatives, non-limiting examples include hydroxypropyl guar, and hydroxypropyl guar hydroxypropyl trimonium chloride.

The thickening polymers may be combined with polyethylene oxide;polypropylene oxide; and POE-PPO copolymers.

The thickening polymers may be combined with polyalkylene glycols characterized by the general formula:

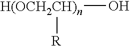

wherein R is hydrogen, methyl, or mixtures thereof, and further hydrogen, and n is an integer having an average from 2,000-180,000, or from 7,000-90,000, or from 7,000-45,000. Non-limiting examples of this class include PEG-7M, PEG-14M, PEG-23M, PEG-25M, PEG-45M, PEG-90M, or PEG-100M.

The thickening polymers may be combined with silicas, non-limiting examples include fumed silica, precipitated silica, and silicone-surface treated silica.

The thickening polymers may be combined with water-swellable clays, non-limiting examples include laponite, bentolite, montmorilonite, smectite, and hectonite.

The thickening polymers may be combined with gums, non-limiting examples include xanthan gum, guar gum, hydroxypropyl guar gum, Arabia gum, tragacanth, galactan, carob gum, karaya gum, and locust bean gum.

The thickening polymers may be combined with, dibenzylidene sorbitol, karaggenan, pectin, agar, quince seed (Cydonia oblonga Mill), starch (from rice, corn, potato, wheat, etc), starch-derivatives (e.g. carboxymethyl starch, methylhydroxypropyl starch), algae extracts, dextran, succinoglucan, and pulleran,

Non-limiting examples of thickening polymers include acrylamide/ammonium acrylate copolymer (and) polyisobutene (and) polysorbate 20; acrylamide/sodium acryloyldimethyl taurate copolymer/isohexadecane/polysorbate 80, ammonium acryloyldimethyltaurate/VP copolymer, Sodium Acrylate/Sodium Acryloyldimethyl Taurate Copolymer, acrylates copolymer, Acrylates Crosspolymer-4, Acrylates Crosspolymer-3, acrylates/beheneth-25 methacrylate copolymer, acrylates/C10-C30 alkyl acrylate crosspolymer, acrylates/steareth-20 itaconate copolymer, ammonium polyacrylate/Isohexadecane/PEG-40 castor oil; carbomer, sodium carbomer, crosslinked polyvinylpyrrolidone (PVP), polyacrylamide/C13-14 isoparaffin/laureth-7, polyacrylate 13/polyisobutene/polysorbate 20, polyacrylate crosspolymer-6, polyamide-3, polyquaternium-37 (and) hydrogenated polydecene (and)trideceth-6, Acrylamide/Sodium Acryloyldimethyltaurate/Acrylic Acid Copolymer, sodium acrylate/acryloyldimethyltaurate/dimethylacrylamide, crosspolymer (and) isohexadecane (and) polysorbate 60, sodium polyacrylate. Exemplary commercially-available thickening polymers include ACULYN™ 28, ACULYN™ 33, ACULYN™ 88, ACULYN™ 22, ACULYN™ Excel, Carbopol® Aqua SF-1, Carbopol® ETD 2020, Carbopol® Ultrez 20, Carbopol® Ultrez 21, Carbopol® Ultrez 10, Carbopol® Ultrez 30, Carbopol® 1342, Carbopol® Aqua SF-2 Polymer, Sepigel™ 305, Simulgel™ 600, Sepimax Zen, Carbopol® SMART 1000, Rheocare® TTA, Rheomer® SC-Plus, STRUCTURE® PLUS, Aristoflex® AVC, Stabylen 30 and combinations thereof.

### Scalp Care Actives

The present invention may comprise a scalp care active. The scalp care active includes soluble scalp care actives and scalp health agents.

### a) Soluble Scalp Care Active

Soluble scalp care active and/or anti-dandruff agent may be one material or a mixture selected from the groups consisting of: azoles, such as climbazole, ketoconazole, itraconazole, econazole, and elubiol; hydroxy pyridones, such as octopirox (piroctone olamine), ciclopirox, rilopirox, and MEA-Hydroxyoctyloxypyridinone; kerolytic agents, such as salicylic acid and other hydroxy acids; strobilurins such as azoxystrobin and metal chelators such as 1,10-phenanthroline.

In the present invention, the azole anti-microbials may be an imidazole selected from the group consisting of: benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and mixtures thereof, or the azole anti-microbials is a triazole selected from the group consisting of: terconazole, itraconazole, and mixtures thereof. The azole anti-microbial agent may be ketoconazole. Further, the sole anti-microbial agent may be ketoconazole.

The soluble anti-dandruff agent may be present in an amount from about 0.01% to 10%, from about 0.1% to about 9%, from about 0.25% to 8%, and from about 0.5% to 6%. The soluble antidandruff agent can be surfactant soluble and thus surfactant soluble antidandruff agents.

### b) Scalp Health Agents

In the present invention, one or more scalp health agent may be added to provide scalp benefits and/or anti-fungal/anti-dandruff efficacy. This group of materials is varied and provides a wide range of benefits including moisturization, barrier improvement, anti-fungal, anti-microbial and anti-oxidant, anti-itch, and sensates, and additional anti-dandruff agents such as polyvalent metal salts of pyrithione, non-limiting examples include zinc pyrithione (ZPT) and copper pyrithione, sulfur, or selenium sulfide. Such scalp health agents include but are not limited to: vitamin E and F, salicylic acid, niacinamide, caffeine, panthenol, zinc oxide, zinc carbonate, basic zinc carbonate, glycols, glycolic acid, PCA, PEGs, erythritol, glycerin, triclosan, lactates, hyaluronates, allantoin and other ureas, betaines, sorbitol, glutamates, xylitols, menthol, menthyl lactate, vanillyl butyl ether, iso cyclomone, benzyl alcohol, a compound comprising the following structure:

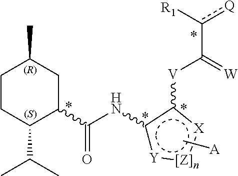

R₁ is selected from H, alkyl, amino alkyl, alkoxy;
Q=H2, O,—OR₁,—N(R₁)₂,—OPO (OR₁)ₓ,—PO (OR₁)ₓ,—P(OR₁) x where x=1-2;
V=NR1, O,—OPO (OR₁)ₓ,—PO(OR₁)ₓ,—P (OR₁)ₓ where x=1-2;
W=H₂, O;
X, Y=independently selected from H, aryl, naphthyl for n=0;
X, Y=aliphatic CH₂ or aromatic CH for n>1 and Z is selected from aliphatic CH₂, aromatic CH, or heteroatom;
A=lower alkoxy, lower alkylthio, aryl, substituted aryl or fused aryl; and
stereochemistry is variable at the positions marked *. and natural extracts/oils including peppermint, spearmint, argan, jojoba and aloe.

The scalp care active may be in an encapsulated form in the present invention. In one aspect, the capsule may comprise: melamine, polyacrylamide, silicones, silica, polystyrene, polyurea, polyurethanes, polyacrylate based materials, polyacrylate esters-based materials, gelatin, styrene malic anhydride, polyamides, aromatic alcohols, polyvinyl alcohol, fatty alcohols, polysaccharides, waxes, hydrogenated vegetable oils and other materials known to those skilled in the art. In one aspect, said polyurea may comprise cross-linked urea, such as urea crosslinked with formaldehyde, urea crosslinked with gluteraldehyde, and mixtures thereof. In one aspect, said polysaccharides may comprise gelatin, agar, alginate, chitosan, cellulose, glycogen, hyaluronic acid, dextran, xylan, inulin, pectin and mixtures thereof. In one aspect, said polysaccharide may be cross-linked. Suitable cross-linkers may comprise calcium chloride, calcium carbonate, isocyanates, glutaraldehyde and mixtures thereof. Typically, ant-dandruff or scalp care actives may be present in an encapsulated form at a concentration from 1% to 5% and even up to 50% weight based on total formula weight or more depending on the chemistry of both the material to be encapsulated and the encapsulate structure itself. In the present invention the composition may contain up to 90% encapsulates, may be up to 10%, may contain up to 5%, may be up to 1%.

In the present invention the personal care composition may be transparent or clear. The term “clear” or “transparent” as used herein, means that the compositions have a percent transparency (% T) of at least about 70% transmittance at 600 nm. The % T may be at 600 nm from about 70% to about 100%, from about 80% to about 100%, from about 90% to about 100%. In the present invention, the percent transparency (% T) may be at least about 80% transmittance at 600 nm; percent transparency (% T) may be at least about 90% transmittance at 600 nm.

In the present invention the personal care composition may be translucent or may be opague. The transparency of the composition is measured by Ultra-Violet/Visible (“UV/VIS”) spectrophotometry, which determines the absorption or transmission of UV/VIS light by a sample, using a Gretag Macbeth Colorimeter Color. A light wavelength of 600 nm has been shown to be adequate for characterizing the degree of clarity of cleansing compositions.

The personal care compositions of the present invention may have from about 14% to about 50% water; may have from about 35% to about 50% water.

### Test Methods for Formulation

### Water Activity

In the present invention, water activity is either measured in Aw (when on a scale of 0-1) or relative humidity-% RH when it is reported as a percentage RH %=aw*100. The water activity (Aw) of the personal care composition is the ratio between the vapor pressure of the personal care composition itself, when in an undisturbed balance with the surrounding air media, and the vapor pressure of distilled water under identical conditions.

### Water Activity Determination

In the present invention, equipment that may be used for Water Activity Determination may be: A) Hygrolab C-1 water activity meter, equipped with temperature and humidity probe (Available from Rotronic AG) and B) Shallow, disposable sample cup (Available from Rotronic AG). In the present invention, the water activity of the test materials may be determined by using a temperature and humidity probe and a Hygrolab C-1 meter (available from Rotronic AG) A disposable sample cup (available from Rotronic AG) is filled with test material, lowered into the sample holder, and covered by the humidity and temperature probe. Using the meter's AwE mode, the water activity of the equilibrated product will be displayed on the meter as water activity (Aw). The following conversion factor can be used to switch between units: 1.000 Aw=100% RH.Viscosity Method.

The present invention may have a Water Activity (Aw) of from about 0.40 to about 0.90; may have a Water Activity (AW) of from about 0.80 to about 0.87. The present invention may have a Water Activity (Aw) that is below from about 0.80.

### Viscosity Determination

In the present invention, equipment and instrumentation that may be used for viscosity determination are: A) Disposable syringe (Available from VWR); Rheometer (Available from TA Instruments) and C) 40 mm parallel steel plates (Available from TA Instruments). In the present invention, the viscosity of the shampoo test materials may be determined by using a Discovery DHR rheometer from TA instruments (New Castle, Delaware, USA). Data collection, processing, and reporting are executed using TRIOS software, version 5.1.1.46572 (available from TA Instruments). The instrument is configured using a 40 mm diameter parallel steel plate, a gap size of 1000 μm, and a temperature of 25° C. The data is collected using a flow peak hold at a shear rate of 2.0 s-1 with a duration of 180 seconds, and the reported viscosity is the value measured at 180 seconds. In the present invention, the personal care composition may have a viscosity of from about 5,000 cps to about 20,000 cps; from about 8,000 cps to about 14,000 cps; from about 7,000 cps to about 12,000 cps.

### Product Reformulation to Reduce Water Activity & Water Content

In the present invention, in order to prevent early hydrolysis of the biodegradable polymeric layer on the inside of the pulp container, it is necessary to reduce both the water activity and water content of the shampoo formulation. This is achieved by modifying a commercial marketed shampoo formulation, by removing any added water that does not come into the formula as part of another ingredient (e.g. surfactants typically come as part of a solution containing water). Because there is a need of the shampoo to be liquid and to have a similar viscosity as commercial marketed shampoo (in order to allow good spread across the hair), only flowable liquids and soluble solids are considered to replace the 29-41% added water in the cosmetic marketed shampoo. Because it is desired to not only reduce the water content, but also reduce the water activity (mobility) of the remaining water in the formula, ingredients that bind water (humectants) are selected. Replacing 29-41% water in the shampoo with a combination of both glycerol and sodium chloride results in a stable formula with both lower water content and activity. In addition, in comparison to water glycerol is known to provide a conditioned feel on hair.

### Non-limiting Examples

The personal care compositions illustrated in the following examples are prepared by conventional formulation and mixing methods. All exemplified amounts are listed as weight percent on an active basis and exclude minor materials such as diluents, preservatives, color solutions, imagery ingredients, botanicals, and so forth, unless otherwise specified. All percentages are based on weight unless otherwise specified.

### Formula Examples

Ex. A and Ex. B are traditional cosmetic formula examples with Higher water activity (Aw) included as control comparisons to Examples C-I which are reduced water activity (Aw) formulae examples of this invention.

### Example Descriptions

A-Higher Aw Traditional shampoo
B-Higher Aw Traditional anti-dandruff shampoo
C-Lower Aw
D-Lower Aw
E-Lower Aw Anti-dandruff
F-Lower Aw Anti-dandruff
G-Lower Aw Alternate Humectant
H-Lower Aw Sulfate-free
I-Lower Aw Sulfate-free

| | Formula Examples | | | | | | | | |
|---|---|---|---|---|---|---|---|---|---|
| | A | B | C | D | E | F | G | H | I |
| Sodium Laureth-1 | 13.2 | 0 | 13.2 | 13.2 | 0 | 12.44 | 0 | 0 | 0 |
| Sulfate¹ | | | | | | | | | |
| Sodium Lauryl Sulfate² | 0 | 6 | 0 | 0 | 6 | 1.5 | 0 | 0 | 0 |
| Sodium Laureth Sulfate | 0 | 6 | 0 | 0 | 6 | 0 | 0 | 0 | 0 |
| n > 13³ | | | | | | | | | |
| Sodium Trtideceth-2 | 0 | 0 | 0 | 0 | 0 | 0 | 18 | 0 | 0 |
| sulfate⁴ | | | | | | | | | |
| Sodium Cocoyl | 0 | 0 | 0 | 0 | 0 | 0 | 0 | 7.8 | 6.0 |
| Isethionate⁵ | | | | | | | | | |
| Cocamidopropyl | 2.4 | 1.8 | 2.4 | 3.5 | 1.8 | 1.2 | 3 | 6.6 | 0 |
| Betaine⁶ | | | | | | | | | |
| Lauramidopropyl | 0 | 0 | 0 | 0 | 0 | 0 | 0 | 0 | 6.15 |
| Betaine ⁷ | | | | | | | | | |
| Cocamide MEA⁸ | 0 | 0.85 | 0 | 0 | 0.85 | 1 | 0 | 0 | 0 |
| Hydroxyethylcellulose⁹ | 0 | 0 | 0 | 0.05 | 0 | 0 | 1 | 0 | 0 |
| Polyquaternium- | 0.12 | 0 | 0.12 | 0.12 | 0 | 0 | 0 | 0.25 | 0 |
| 6(DADMAC)¹⁰ | | | | | | | | | |
| Guar | 0.35 | 0.25 | 0.35 | 0.35 | 0.25 | 0.3 | 0 | 0 | 0 |
| Hydroxypropyltrimonium | | | | | | | | | |
| Chloride ¹¹ | | | | | | | | | |
| Polyquaternium-76¹² | 0 | 0 | 0 | 0 | 0 | 0.1 | 0 | 0 | 0 |
| Dimethicone ¹³ | 2.23 | 1.45 | 2.23 | 2.23 | 1.45 | 2.23 | 0 | 1.5 | 0 |
| Climbazole¹⁴ | 0 | 0.5 | 0 | 0 | 0.5 | 0 | 0 | 1 | 0 |
| Piroctone Olamine¹⁵ | 0 | 0 | 0 | 0 | 0 | 0.5 | 0 | 0 | 0 |
| Benzyl Alcohol¹⁶ | 0 | 0.5 | 0 | 0 | 0.5 | 0 | 0 | 0 | 0 |
| Sodium Citrate¹⁷ | 0 | 0 | 0 | 0 | 0 | 0 | 0 | 0.78 | 0 |
| Sodium Chloride¹⁸ | 0.1 | 0.1 | 6 | 4 | 4 | 4 | 6 | 4 | 0 |
| Sodium Salicyate¹⁹ | 0 | 0 | 0 | 0 | 0 | 0 | 0 | 0.45 | 0 |
| Sodium Benzoate²⁰ | 0.25 | 0.25 | 0.25 | 0.25 | 0.25 | 0.25 | 0.25 | 0.8 | 0.45 |
| Methylchloroisothiazolinone/ | 0.0005 | 0.0005 | 0.0005 | 0.0005 | 0.0005 | 0.0005 | 0.0005 | 0 | 0 |
| Methylisothiazolinone²¹ | | | | | | | | | |
| Citric Acid²² | 0.3 | 0.5 | 0.3 | 0.3 | 0.5 | 0.5 | 0.5 | 0.1 | 0.10 |
| Glycol Distearate²³ | 1.4 | 1.5 | 1.4 | 1.4 | 1.5 | 1.5 | 0 | 0 | 0 |
| Stearyl Alcohol²⁴ | 0.64 | 0 | 0.64 | 0.64 | 0 | 0 | 0 | 0 | 0 |
| Cetyl Alcohol²⁵ | 0.32 | 0 | 0.32 | 0.32 | 0 | 0 | 0 | 0 | 0 |
| Dipropyene Glycol²⁶ | 0 | 0 | 0 | 0 | 0 | 0 | 24 | 0 | 0 |
| Glycerin²⁷ | 0 to 1 | 0 | 23.74 | 22.66 | 44.5 | 38.51 | 0 | 40 | 69.98 |
| Fragrance | 0.9 | 1.2 | 1 | 1 | 1.2 | 1 | 1 | 1.2 | 1.0 |
| Water, QS | 77.79 | 79.09 | 48.05 | 49.98 | 30.70 | 34.97 | 46.25 | 35.52 | 14.09 |
| Final Visc, cps | 10000 | 5000 | 14000 | 14000 | 7000 | 12000 | 5000 | 8100 | 13000 |
| Water Activity, Aw | 0.98 | 0.98 | 0.82 | 0.85 | 0.70 | 0.74 | 0.80 | 0.75 | 0.40 |

Ingredients code:

| | |
|---|---|
| 1 | Sodium Laureth-1 Sulfate at 26% active, supplier: P&G |
| 2 | Sodium Lauryl Sulfate at 29% active, supplier: P&G |
| 3 | Sodium Alkyl Ether Sulfate, 28% active, supplier P&G |
| 4 | Sodium Trideceth-2 Sulfate45% active, supplier P&G |
| 5 | HOSTAPON SCI 85 C, supplier Clariant |
| 6 | Tego Betain L 7 OK at 30% active, supplier: Evonik |
| 7 | Lauromidopropyl Betaine Mackam DAB ULS, supplier: Solvay |
| 8 | Ninol Comf at 85% active, supplier: Stepan |
| 9 | Natrasol 250 HHX, supplier Ashland |
| 10 | Flocare C106, supplier: SNF |
| 11 | N-HANCE 3196 Guar Guml, supplier: Ashland |
| 12 | Mirapol 100 at 31.5% active, supplier: SNF, Inc. |
| 13 | CF330M, supplier: Momentive |
| 14 | Crinipan AD, supplier: Symrise |
| 15 | Octopirox, supplier: Clariant |
| 16 | Benzyl Alcohol, supplier: Valtris |
| 17 | Sodium Citrate Dihydrate, supplier: ADM |
| 18 | Sodium Chloride, supplier: Morton |
| 19 | Sodium Salicylate, supplier: Spectrum Chemical |
| 20 | Sodium Benzoate Dense NF/FCC, |
| | supplier: Emerald Performance Materials |
| 21 | Kathon CG at 1.5% active, supplier: Dow |
| 22 | Citric Acid Anhydrous, supplier: ADM; |
| | level adjustable to achieve target pH |
| 23 | TEGIN G 1100, supplier: Evonik |
| 24 | Stearyl Alcohol supplier: P&G Chemicals |
| 25 | Cetyl Alcohol supplier: P&G Chemicals |
| 26 | Dipropylene Glycol, supplier: BASF |
| 27 | Glycerin, supplier: P&G |

Biodegradable Container with Lower Aw Shampoo Stability Data TABLE OF

### Example

| Example | Biodegradable container |
|---|---|
| Wall stock | Pulp, 600 gsm. 3 layers |
| | coating (whole surface |
| | coated), 150 gsm; Primer: |
| | MFC, Top-coat1: |
| | Carnauba wax and linseed |
| | oil, Top-coat 2: Shellac. |
| | 5.8 g |
| Fiber % wall stock | 66% |
| Lidding film | Parkside. HCPT1(b) |
| | Park-2-Nature Home |
| | Compostable Triplex Laminate. |
| | Lacquer/Ink/25 gsm |
| | Paper/Adhesive/23mm |
| | Futamura NM/Adhesive/ |
| | 30 mm Bio-PBS FD92 |
| Leakage and Pinholing | Passed |
| WVTR wall stock, | 21.5 ± 2.9 |
| g/sqm/day at 38° C. | |
| 90% RH (ASTM E96) | |
| Weight Loss wall stock, | 6.6 ± 0.8 |
| g/sqm/day at 25° C. 60% RH | |
| Formula Ex. D (Aw = 85%) | |
| Weight Loss wall stock, | 93.8 ± 5.6 |
| g/sqm/day at 25° C. 60% RH | |
| Formula Ex. A (Aw = 97%) | |
| WVTR lid, g/sqm/day | 13.8 ± 6.6 |
| at 38° C. 90% RH | |
| (ASTM F1249) | |
| Weight Loss lid, | 0.26 +/− 0.03 |
| g/sqm/day at 40C, 75% RH | |
| Formula Ex. D (Aw = 85%) | |
| Weight Loss lid, | 1.45 +/− 0.03 |
| g/sqm/day at 40C, | |
| 75% RH Formula | |
| Ex. A (Aw = 97%) | |
| Biodegradable | Yes |
| Paper recyclability | 59 ± 5 % (wall stock) |
| (PTS-RH-021/97 cat 2) | |
| Disintegrability (fiber | |
| recovery %, target 80%) | |
| Undisturbed sheet formation | |

Biodegradable Container with Lower Aw Shampoo Stability Data

The Table above discloses an exemplary biodegradable container including a biodegradable vessel (wall stock) and exemplary lidding film based on this disclosure. The vessel is wet pulp molded and covered on the whole surface with a liquid containment barrier. The liquid containment barrier is made by three different distinguished layers all applied by dip coating: (1) a first layer including MFC, (2) a second layer including carnauba wax and linseed oil and a (3) third layer including shellac. The lidding film is a home compostable triplex paper laminate commercialized by ParksideÒ. Both vessels and lidding film are found free of pinholes. The whole package is biodegradable. The cup is also found 10 to meet the minimum pass requirements for re-pulpability according to the PTS-RH 021/97 cat 2 method. Both the exemplary vessels and lidding films are individually tested with both a lower water activity (Aw) shampoo Formula Ex.D and a higher water activity (Aw) and measured for weight change over time using Method #9 Weight Loss Test Method as an indication of relative stability. For both the exemplary vessels and lidding films the weight change is found to be significantly higher for the higher Aw traditional shampoo Ex. A than the lower Aw shampoo Ex. D at parity of storage conditions.

### Additional Examples/Combinations

A. A biodegradable container in combination with a liquid personal care composition comprising:
   a) a biodegradable vessel having at least one opening and a biodegradable cover for the opening wherein the container;
   b) a liquid personal care composition comprising from about 14% to about 50% water; from about 20% to about 70% of a humectant; wherein there is a Water Activity (Aw) of about 0.40 to about 0.90.
B. The biodegradable container in combination with a liquid personal care composition according to Paragraph A, wherein the biodegradable vessel comprises cellulose fibers and is pulp molded.
C. The biodegradable container in combination with a liquid personal care composition according to Paragraph A-B, wherein the biodegradable cover is a biodegradable lidding film.
D. The biodegradable container in combination with a liquid personal care composition according to Paragraph A-C, wherein the biodegradable vessel comprises a biodegradable liquid containment barrier with one or more layers and one or more coatings.
E. The biodegradable container in combination with a liquid personal care composition according to Paragraph A-D, wherein the biodegradable vessel comprises an inorganic barrier layer applied on the cellulose fibers by vapor phase deposition after pulp molding.
F. The biodegradable container in combination with a liquid personal care composition according to Paragraph A-E, wherein the biodegradable liquid containment barrier comprises one or more primer layers comprising a material selected from the group consisting of cellulose fibers, polyvinyl alcohol (PVOH), polyvinyl alcohol copolymers, polyhydroxyalkanoates (PHA), polybutylene succinate adipate (PBSA), chitosan, natural gums such as xanthan and carrageenan gum, psyllium husk, sodium alginate, maltodextrin, polysaccharides, casein, whey, agar-agar, thermoplastic starch and mixtures therein.
G. The biodegradable container in combination with a liquid personal care composition according to Paragraph A-F, wherein the biodegradable liquid containment barrier comprises an inorganic barrier layer.
H. The biodegradable container in combination with a liquid personal care composition according to Paragraph A-G, wherein the biodegradable liquid containment barrier comprises one or more topcoat layers selected from the group consisting of linseed oil, carnauba, beeswax, cutin, rapeseed wax, castor wax, candelilla wax, soy wax, palm oil wax, biodegradable paraffin oil-based waxes, shellac, chitosan, inorganic-organic hybrid-polymer and mixtures thereof.
I. The biodegradable container in combination with a liquid personal care composition according to Paragraph A-H, wherein a topcoat free surface energy is between 40 and 50 mN/m and wherein the dispersion energy component is between 32 to 37 mN/m the polar energy component is between 5 to 16 mN/m.
J. The biodegradable container in combination with a liquid personal care composition according to Paragraph A-I, wherein the liquid containment barrier is a thermoplastic material selected from the group consisting of aliphatic and/or aromatic polyesters or thermoplastic starches.
K. The biodegradable container in combination with a liquid personal care composition according to Paragraph A-J, wherein the thermoplastic material is applied by hot vacuum thermoforming.
L. The biodegradable container in combination with a liquid personal care composition according to Paragraph A-K, wherein the biodegradable vessel is made from a biodegradable paper board.
M. The biodegradable container in combination with a liquid personal care composition according to Paragraph A-L, wherein both the biodegradable vessel and biodegradable cover are pulp molded.
N. The biodegradable container in combination with a liquid personal care composition according to Paragraph A-M, wherein the biodegradable vessel and the biodegradable cover are pulp molded in one piece and comprise a hinge.
O. The biodegradable container in combination with a liquid personal care composition according to Paragraph A-N, wherein the biodegradable lidding film comprises a sealant layer made from a water in-soluble biodegradable polymer.
P. The biodegradable container in combination with a liquid personal care composition according to Paragraph A-O, wherein the biodegradable lidding film comprises an inorganic barrier layer.
Q. The biodegradable container in combination with a liquid personal care composition according to Paragraph A-P, wherein the biodegradable lidding film comprises cellulose.
R. The biodegradable container in combination with a liquid personal care composition according to Paragraph A-Q, wherein the biodegradable vessel comprises a component grafted to the biodegradable vessel wherein the component is made of a biodegradable polymer.
S. The biodegradable container in combination with a liquid personal care composition according to Paragraph A-R, wherein the biodegradable polymer is a thermoplastic starch.
T. The biodegradable container in combination with a liquid personal care composition according to Paragraph A-S, wherein the liquid personal care composition comprises from about 35% to about 50% water.
U. The biodegradable container in combination with a liquid personal care composition according to Paragraph A-T, wherein the humectant is from about 23% to about 45%.
V. The biodegradable container in combination with a liquid personal care composition according to Paragraph A-U, wherein the humectant is selectant from the group consisting of glycerin, amino acids, proline, arginine aspartate 1,3-butylene glycol, propylene glycol and water, codium tomentosum extract, collagen amino acids or peptides, creatinine, diglycerol, biosaccharide gum-1, glucamine salts, glucuronic acid salts, glutamic acid salts, polyethylene glycol ethers of glycerine, glycerine, glycerol monopropoxylate, glycogen, hexylene glycol, honey, hydrogenated starch hydrolysates, hydrolyzed mucopolysaccharides, inositol, keratin amino acids, glycosaminoglycans, methoxy PEG 10, methyl gluceth-10 and −20, methyl glucose, 3-methyl-1,3-butanediol, N-acetyl glucosamine salts, polyethylene glycol, PEG 4, PEG 5 pentaerythitol, PEG 6, PEG 8, PEG 9, pentaerythitol, 1,2 pentanediol, PPG-1 glyceryl ether, PPG-9,2-pyrrolidone-5-carboxylic acid and salts, glyceryl pca, saccharide isomerate, sericin, silk amino acids, sodium acetylhyaluronate, sodium hyaluronate, sodium poly-aspartate, sodium polyglutamate, sorbeth 20, sorbeth 6, sugar and sugar alcohols, glucose, sucrose, fructose, mannose, polyglycerol sorbitol, trehalose, triglycerol, trimethyolpropane, tris(hydroxymethyl) amino methane salts, yeast extract, ionic salts, sodium chloride potassium chloride and mixtures thereof.
W. The biodegradable container in combination with a liquid personal care composition according to Paragraph A-V, wherein the humectant is selected from the group consisting of glycerin, sodium chloride and mixtures thereof.
X. The biodegradable container in combination with a liquid personal care composition according to Paragraph A-W, wherein the Water Activity (Aw) is from about 0.80 to about 0.90.
Y. The biodegradable container in combination with a liquid personal care composition according to Paragraph A-X, wherein the Water Activity (Aw) is below from about 0.80.
Z. The biodegradable container in combination with a liquid personal care composition according to Paragraph A-Y, wherein the liquid personal care composition comprises a surfactant.
AA. The biodegradable container in combination with a liquid personal care composition according to Paragraph A-Z, wherein the surfactant is from about 10% to about 18%.
BB. The biodegradable container in combination with a liquid personal care composition according to Paragraph A-AA, wherein the surfactant is selected from one or more anionic surfactants.
CC. The biodegradable container in combination with a liquid personal care composition according to Paragraph A-BB, wherein the liquid personal care composition comprises an amphoteric co-surfactant.
DD. The biodegradable container in combination with a liquid personal care composition according to Paragraph A-CC, wherein the amphoteric co-surfactant is selected from the group consisting of cocamidopropyl betaine, lauramidopropyl betaine and mixtures thereof.
EE. The biodegradable container in combination with a liquid personal care composition according to Paragraph A-DD, wherein the liquid personal care composition comprises a thickener.
FF. The biodegradable container in combination with a liquid personal care composition according to Paragraph A-EE, wherein the liquid personal care composition comprises a thickener selected from the group consisting of acrylic acid/acrylonitrogens copolymer, acrylates/steareth-20 itaconate copolymer, acrylates/ceteth-20 itaconate copolymer, Acrylates/Aminoacrylates/C10-30 Alkyl PEG-20 Itaconate Copolymer, acrylates/aminoacrylates copolymer, acrylates/steareth-20 methacrylate copolymer, acrylates/beheneth-25 methacrylate copolymer, acrylates/steareth-20 methacrylate crosspolymer, acrylates/beheneth-25 methacrylate/HEMA crosspolymer, acrylates/vinyl neodecanoate crosspolymer, acrylates/vinyl isodecanoate crosspolymer, Acrylates/Palmeth-25 Acrylate Copolymer, Acrylic Acid/Acrylamidomethyl Propane Sulfonic Acid Copolymer, and acrylates/C10-C30 alkyl acrylate crosspolymer and mixtures thereof.
GG. The biodegradable container in combination with a liquid personal care composition according to Paragraph A-FF, wherein the thickener is selected from the group consisting of hydroxyethyl cellulose.
HH. The biodegradable container in combination with a liquid personal care composition according to Paragraph A-GG, wherein the liquid personal care composition comprises sodium chloride.
II. The biodegradable container in combination with a liquid personal care composition according to Paragraph A-HH, wherein the liquid personal care composition comprises a scalp health active.
JJ. The biodegradable container in combination with a liquid personal care composition according to Paragraph A-II, wherein the scalp health active is selected from the group consisting of piroctone olamine, zinc pyrithione, climbazole, sulfur and mixtures thereof.
KK. The biodegradable container in combination with a liquid personal care composition according to Paragraph A-JJ, wherein the personal care composition has a viscosity of from about 5,000 cps to about 20,000 cps.
LL. The biodegradable container in combination with a liquid personal care composition according to Paragraph A-KK, wherein the liquid personal care composition gains water when packaged inside the container, wherein the liquid personal care composition gain of water enables physical or chemical properties of a final formulation of the liquid personal care composition.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention

What is claimed is:

1. A biodegradable container in combination with a liquid personal care composition comprising:
a) a biodegradable vessel having at least one opening and a biodegradable cover for the opening;
b) a liquid personal care composition comprising from about 14% to about 50% water; from about 20% to about 70% of a humectant; wherein there is a Water Activity (Aw) of about 0.40 to about 0.90.

2. A biodegradable container according to claim 1, wherein the biodegradable vessel comprises cellulose fibers and is pulp molded.

3. A biodegradable container according to claim 1, wherein the biodegradable cover is a biodegradable lidding film.

4. A biodegradable container according to claim 2, wherein the biodegradable vessel comprises a biodegradable liquid containment barrier with one or more layers and one or more coatings.

5. A biodegradable container according to claim 2, wherein the biodegradable vessel comprises an inorganic barrier layer applied on the cellulose fibers by vapor phase deposition after pulp molding.

6. A biodegradable container according to claim 4, wherein the biodegradable liquid containment barrier comprises one or more primer layers comprising a material selected from the group consisting of cellulose fibers, polyvinyl alcohol (PVOH), polyvinyl alcohol copolymers, polyhydroxyalkanoates (PHA), polybutylene succinate adipate (PBSA), chitosan, natural gums such as xanthan and carrageenan gum, psyllium husk, sodium alginate, maltodextrin, polysaccharides, casein, whey, agar-agar, thermoplastic starch and mixtures therein.

7. A biodegradable container according to claim 4, wherein the biodegradable liquid containment barrier comprises an inorganic barrier layer.

8. A biodegradable container according to claim 4, wherein the biodegradable liquid containment barrier comprises one or more topcoat layers selected from the group consisting of linseed oil, carnauba, beeswax, cutin, rapeseed wax, castor wax, candelilla wax, soy wax, palm oil wax, biodegradable paraffin oil-based waxes, shellac, chitosan, inorganic-organic hybrid-polymer and mixtures thereof.

9. A biodegradable container according to claim 8, wherein a topcoat free surface energy is between 40 and 50 mN/m and wherein the dispersion energy component is between 32 to 37 mN/m the polar energy component is between 5 to 16 mN/m.

10. A biodegradable container according to claim 4, wherein the liquid containment barrier is a thermoplastic material selected from the group consisting of aliphatic and/or aromatic polyesters or thermoplastic starches.

11. A biodegradable container according to claim 10 wherein the thermoplastic material is applied by hot vacuum thermoforming.

12. A biodegradable container according to claim 1, wherein the biodegradable vessel is made from a biodegradable paper board.

13. A biodegradable container according to claim 1, wherein both the biodegradable vessel and biodegradable cover are pulp molded.

14. A biodegradable container according to claim 13, wherein the biodegradable vessel and the biodegradable cover are pulp molded in one piece and comprise a hinge.

15. A biodegradable container according to claim 3, wherein the biodegradable lidding film comprises a sealant layer made from a water in-soluble biodegradable polymer.

16. A biodegradable container according to claim 3, wherein the biodegradable lidding film comprises an inorganic barrier layer.

17. A biodegradable container according to claim 3, wherein the biodegradable lidding film comprises cellulose.

18. A biodegradable container according to claim 1, wherein the biodegradable vessel comprises a component grafted to the biodegradable vessel wherein the component is made of a biodegradable polymer.

19. A biodegradable container according to claim 18, wherein the biodegradable polymer is a thermoplastic starch.

20. A biodegradable container according to claim 1 wherein the liquid personal care composition comprises from about 35% to about 50% water.

21. A biodegradable container according to claim 1 wherein the humectant is from about 23% to about 45%.

22. A biodegradable container according to claim 1 wherein the humectant is selectant from the group consisting of glycerin, amino acids, proline, arginine aspartate 1,3-butylene glycol, propylene glycol and water, codium tomentosum extract, collagen amino acids or peptides, creatinine, diglycerol, biosaccharide gum-1, glucamine salts, glucuronic acid salts, glutamic acid salts, polyethylene glycol ethers of glycerine, glycerine, glycerol monopropoxylate, glycogen, hexylene glycol, honey, hydrogenated starch hydrolysates, hydrolyzed mucopolysaccharides, inositol, keratin amino acids, glycosaminoglycans, methoxy PEG 10, methyl gluceth-10 and −20, methyl glucose, 3-methyl-1,3-butanediol, N-acetyl glucosamine salts, polyethylene glycol, PEG 4, PEG 5 pentaerythitol, PEG 6, PEG 8, PEG 9, pentaerythitol, 1,2 pentanediol, PPG-1 glyceryl ether, PPG-9,2-pyrrolidone-5-carboxylic acid and salts, glyceryl pca, saccharide isomerate, sericin, silk amino acids, sodium acetylhyaluronate, sodium hyaluronate, sodium poly-aspartate, sodium polyglutamate, sorbeth 20, sorbeth 6, sugar and sugar alcohols, glucose, sucrose, fructose, mannose, polyglycerol sorbitol, trehalose, triglycerol, trimethyolpropane, tris(hydroxymethyl) amino methane salts, yeast extract, ionic salts, sodium chloride potassium chloride and mixtures thereof.

23. A biodegradable container according to claim 22 wherein the humectant is selected from the group consisting of glycerin, sodium chloride and mixtures thereof.

24. A biodegradable container according to claim 1 wherein the Water Activity (Aw) is from about 0.80 to about 0.90.

25. A biodegradable container according to claim 1 wherein the Water Activity (Aw) is below from about 0.80.

26. A biodegradable container according to claim 1 wherein the liquid personal care composition comprises a surfactant.

27. A biodegradable container according to claim 26 wherein the surfactant is from about 10% to about 18%.

28. A biodegradable container according to claim 26 wherein the surfactant is selected from one or more anionic surfactants.

29. A biodegradable container according to claim 1 wherein the liquid personal care composition comprises an amphoteric co-surfactant.

30. A biodegradable container according to claim 29 wherein the amphoteric co-surfactant is selected from the group consisting of cocamidopropyl betaine, lauramidopropyl betaine and mixtures thereof.

31. A biodegradable container according to claim 1 wherein the liquid personal care composition comprises a thickener.

32. A biodegradable container according to claim 31 wherein the liquid personal care composition comprises a thickener selected from the group consisting of acrylic acid/acrylonitrogens copolymer, acrylates/steareth-20 itaconate copolymer, acrylates/ceteth-20 itaconate copolymer, Acrylates/Aminoacrylates/C10-30 Alkyl PEG-20 Itaconate Copolymer, acrylates/aminoacrylates copolymer, acrylates/steareth-20 methacrylate copolymer, acrylates/beheneth-25 methacrylate copolymer, acrylates/steareth-20 methacrylate crosspolymer, acrylates/beheneth-25 methacrylate/HEMA crosspolymer, acrylates/vinyl neodecanoate crosspolymer, acrylates/vinyl isodecanoate crosspolymer, Acrylates/Palmeth-25 Acrylate Copolymer, Acrylic Acid/Acrylamidomethyl Propane Sulfonic Acid Copolymer, and acrylates/C10-C30 alkyl acrylate crosspolymer and mixtures thereof.

33. A biodegradable container according to claim 32 wherein the thickener is selected from the group consisting of hydroxyethyl cellulose.

34. A biodegradable container according to claim 1 wherein the liquid personal care composition comprises sodium chloride.

35. A biodegradable container according to claim 1 wherein the liquid personal care composition comprises a scalp health active.

36. A biodegradable container according to claim 35 wherein the scalp health active is selected from the group consisting of piroctone olamine, zinc pyrithione, climbazole, sulfur and mixtures thereof.

37. A biodegradable container according to claim 1 wherein the personal care composition has a viscosity of from about 5,000 cps to about 20,000 cps.

38. A biodegradable container in combination with a liquid personal care composition wherein the liquid personal care composition gains water when packaged inside the container, wherein the liquid personal care composition gain of water enables physical or chemical properties of a final formulation of the liquid personal care composition.
